(19) United States
(12) Patent Application Publication
(10) Pub. No.: US 2005/0037423 A1
(43) Pub. Date: Feb. 17, 2005

(54) Isotopycally coded affinity marker 2

(75) Inventors: Hans-Georg Lerchen, Leverkusen (DE); Oswald Lockhoff, Leverkusen (DE); Dorian Immler, Leverkusen (DE); Hans-Ulrich Siegmund, Leverkusen (DE)

Correspondence Address: JEFFREY M. GREENMAN, BAYER PHARMACEUTICALS CORPORATION, 400 MORGAN LANE, WEST HAVEN, CT, 06516, US

(73) Assignee: Bayer Healthcare AG, Leverkusen (DE)

(21) Appl. No.: 10/494,748

(22) PCT Filed: Oct. 30, 2002
(86) PCT No.: PCT/EP02/12106

(30) Foreign Application Priority Data
Nov. 9, 2001 (DE) 101 54 753.6
Jul. 29, 2002 (DE) 102 34 416.7

(51) Int. Cl. G01N 33/53; C07K 14/00

(52) U.S. Cl. 435/7.1; 530/408

(57) ABSTRACT

The invention concerns isotopically coded marker (ICAT) for mass spectrometric analysis of proteins, and the preparation and use of said markers.

The invention relates to novel, isotope-coded affinity tags for the mass-spectrometric analysis of proteins, and to their preparation and use.

Proteomics technology opens up the possibility of identifying novel biological targets and tags by means of analyzing biological systems at the protein level. It is known that only a certain proportion of all the possible proteins encoded in the genome is being expressed at any given time, with, for example, tissue type, state of development, activation of receptors or cellular interactions influencing the pattern and rates of expression. In order to detect differences in the expression of proteins in healthy or diseased tissue, it is possible to make use of a variety of comparative methods for analyzing protein expression patterns ((a) S. P. Gygi et al., Proc. Natl. Acad. Sci. U. S. A., 2000, 97, 9390; (b) D. R. Goodlett et al., Proteome Protein Anal., 2000, 3; (c) S. P. Gygi et al., Curr. Opin. Biotechnol., 2000, 11, 396).

The mass-spectrometric detection of proteins is a powerful method in this connection. When affinity tags which have been isotope-coded differently (ICAT® =isotope coded affinity lags) and tandem mass spectrometry are used, this method can be enlisted for quantitatively analyzing complex protein mixtures ((a) S. P. Gygi et al., Nature Biotechnology, 1999, 17, 994; (b) R. H. Aebersold et al., WO 00/11208). The method is based on each of two or more protein mixtures, which are to be compared and which have been obtained in different cell states, being reacted with an affinity tag of a different isotope coding. After that, the protein mixes are combined, where appropriate fractionated or treated proteolytically and purified by affinity chromatography. After the bound fragments have been eluted, the eluates are analyzed by a combination of liquid chromatography and mass spectrometry (LC-MS). Pairs or groups of peptides which are labeled with affinity tags which only differ in the isotope coding are chemically identical and are eluted virtually simultaneously in the HPLC; however, they differ in the mass spectrometer by the respective molecular weight differences due to the affinity tags having different isotope patterns. Relative protein concentrations can be obtained directly by carrying out measurements of the peak areas. Suitable affinity tags are conjugates composed of affinity ligands which are linked covalently to protein-reactive groups by way of bridge members. In connection with this, different isotopes are incorporated into the bridge members. The method was described using affinity tags in which hydrogen atoms were replaced with deuterium atoms (¹H/²D isotope coding).

The method using ¹H/²D isotope-coded affinity tags which is described in the prior art suffers from a variety of disadvantages, in particular an isotope effect of the differently labeled, but otherwise identical, peptide fragments in the LC, inadequate stability of the affinity tags in general and especially in LC-MS/MS, and a lack of efficiency as regards the avidin monomer-based affinity chromatography.

The object of the present invention was to make available improved affinity tags.

The invention relates to organic compounds which are suitable for use as affinity tag reagents for the mass-spectrometric analysis of proteins which are of the formula (I),
A-L-PRG (I)
in which
A is an affinity ligand residue,
PRG is a protein-reactive group, and
L is a linker which covalently links A and PRG,
where the linker L contains an acid-cleavable group S of the formula 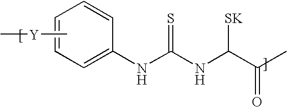
   in which
Y is an optionally branched spacer group having a chain length of from 1 to 10, preferably of from 1 to 5, non-hydrogen atoms, and
SK is the side chain of an amino acid,
or the salts thereof.

The invention furthermore relates to the use of one or more differently isotope-labeled compounds according to the invention as (a) reagent(s) for the mass-spectrometric analysis of proteins, in particular for identifying one or more proteins or protein functions in one or more protein-containing samples and for determining the relative level of expression of one or more proteins in one or more protein-containing samples.

As a predetermined breaking point, the acid-cleavable group S ensures that the affinity tag is cleaved under the influence of acid in order, in this way, for example, to facilitate release from the affinity column, to decrease the size of the residue remaining on the peptide and/or to make the operational procedures more efficient overall. In the case of biotin-modified peptide fragments, this acid-labile predetermined breaking point makes it possible to decomplex the peptide fragments using what is a substantially more efficient straptavidin-based affinity chromatography. It is also advantageous that the tags which remain on the peptide fragments following acid cleavage have a markedly lower molecular weight and a higher isotope density. Furthermore, the manipulation of the affinity tags is improved, as compared with the prior art, as a result of superior solubility and as a result of a crystalline or amorphous nature.

The spacer group Y, which is contained in the acid-cleavable group S, is bonded to the benzene ring in the ortho, meta or para position in relation to the nitrogen atom, with the para position being preferred. Examples of suitable spacer groups Y are chains which are constructed from the building blocks NH, CH₂ and/or CO and in which one or more hydrogen atoms can be substituted by identical or different, optionally heteroatom-containing hydrocarbon radicals, in particular C₁-C₄-alkyl radicals. Y preferably contains at least one NH group, in particular at its end facing away from the benzene ring. Particularly preferred spacer groups Y are NH, NH—CH₂ and NH—CH₂—CH₂—NH—CO, with the latter two preferably being bonded to the benzene ring by the CH₂ and CO groups, respectively.

The amino acid side chain SK is the side chain of an α-amino acid of the formula SK—CH(NH₂)—COOH, which, in the case of SKs other than an H atom, can be present in the D or L form or in racemic form. Examples of suitable SKs are the side chains of the 20 natural amino acids and their D forms and racemates, e.g. the side chains of L-glycine, L-histidine, L-valine, D-valine, L-proline, L-asparagine, L-aspartic acid and L-glutamic acid. Other functional groups which may possibly be present in SKs, for example in the case of amino acids such as histidine or aspartic acid, can optionally be present in free form or be protected with a protecting group.

The affinity ligand A is used for selectively enriching samples by means of affinity chromatography. The affinity columns are provided with the corresponding reactants which are complementary to the affinity ligands, which reactants enter into covalent or noncovalent bonds with the affinity ligands. An example of a suitable affinity ligand is biotin or a biotin derivative, which enters into strong, noncovalent bonds with the complementary peptides avidin or steptavidin. In this way, it is possible to use affinity chromatography to selectively isolate samples to be investigated from sample mixtures. In the same sense, it is also possible for example, to use carbohydrate residues, which are able to enter into noncovalent interactions with fixed lectins, for example, as affinity ligands. Furthermore, the interaction of haptens with antibodies or the interaction of transition metals with corresponding ligands, as sequestering agents, can be used in the same sense, as can other systems which interact with each other. In yet another embodiment, the affinity ligand A can be a functional group which enables the affinity tag reagent to be covalently fixed to a polymeric matrix.

In a preferred embodiment of the compounds according to the invention, A is the acyl residue of an affinity ligand, for example biotinyl or a biotin derivative.

Protein-reactive groups PRGs are used for selectively labeling the proteins at selected functional groups. PRGs have a specific reactivity for terminal functional groups in the proteins. Examples of amino acids which, as elements of proteins, are frequently used for selective labelings, are mercaptoaminomonocarboxylic acids, such as cysteine, diaminomonocarboxylic acids, such as lysine or arginine, or monoaminodicarboxylic acids, such as aspartic acid or glutamic acid. Furthermore, protein-reactive groups can also be phosphate-reactive groups, such as metal chelates, and also aldehyde-reactive and ketone-reactive groups, such as amines with sodium borohydride or sodium cyanoborohydride. They can also be groups which, following selective protein derivatization, such as a cyanogen bromide cleavage or an elimination of phosphate groups, etc., react with the products of the reaction.

In a preferred embodiment of the compounds according to the invention, PRG is the residue of a protein-reactive group, which group is characterized by an electrophilic group and a suitable bridge which permits or facilitates the binding of the electrophilic group to the linker L, preferably bridged electrophiles such as 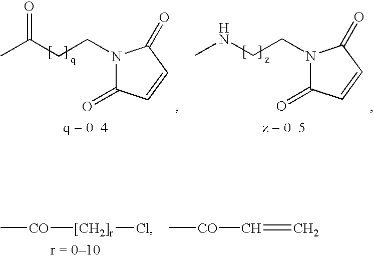
or another known protein-reactive group as are described and summarized, for example, by W. H. Scouten in Methods in Enzymology, Volume 135, edited by Klaus Mosbach, Academic Press Inc. 1987, pp. 30 ff.

In order to improve the solubility of the affinity tags according to the invention, acid and/or basic functional groups which are present can be prepared and employed in the form of their salts, preferably their hydrochlorides, trifluoroacetates or alkali metal salts.

In one particular embodiment of the invention, the protein-reactive group PRG possesses solubility-improving functional groups.

Preferred compounds according to the invention are those of the formula (II)
A-S—B¹—X¹—(CH₂)ₙ—[X²—(CH₂)ₘ]ₓ—X³—(CH₂)ₚ—X⁴—PRG (II)
in which
A is the acyl residue of an affinity ligand, for example biotinyl or a biotin derivative,
PRG is the residue of a protein-reactive group, which group is characterized by an electrophilic group and a suitable bridge which permits or facilitates the binding of the electrophilic group to X⁴, preferably bridged electrophiles such as 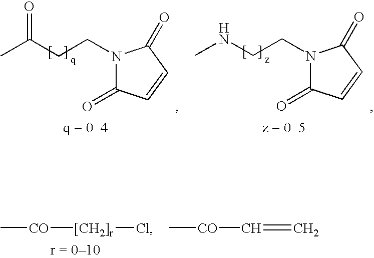
   or another known protein-reactive group as are described and summarized, for example, by W. H. Scouten in Methods in Enzymology, Volume 135, edited by Klaus Mosbach, Academic Press Inc. 1987, pp. 30 ff,
S is the acid-cleavable group according to the invention,
X¹, X² and X³ are, independently of each other, and also, in the case of X², independently of other X², in each case O, S, NH, NR, CO, CO—O, O—CO, CO—S, S—CO, S—S, SO, SO₂, CO—NR, NR—CO, CS—NR, NR—CS, Si—O or O—Si or arylene or diarylene groups, where X¹, X² or X³ may also be partially or completely absent,
X⁴ is O, S, NH, NR, CO—NR, NR—CO, CS—NR or NR—CS or arylene or diarylene groups, particularly preferably NH, NR, O or S,
m, n, p, q, r, z, s and x are, independently of each other, in each case a number from 0 to 100, where the sum n+xm+p is preferably less than 100 and particularly preferably between 5 and 30,
B¹ is an optionally present amine group NRR′ having the connectivity S—NRR′—X¹, which, as a bridge, links S to X¹, where B¹ together with X¹ can be part of an amino acid derivative, and
R and R′ are, independently of each other, in each case hydrogen or alkyl, alkenyl, alkynyl, alkoxy or aryl, or are both, together with N, an N heterocycle NRR′, where R′ is additionally bonded to X¹ and cannot therefore be hydrogen, where R and R′, as well as X¹, can furthermore be modified with functional groups which, for example, improve the solubility, such as carboxyl or amino groups, and where R and R′ can be substituted in the form such that B¹—X¹ together result in the derivative of an amino acid and where the functional groups which are not involved in the bonding with S or with CH₂ contribute, for example, to the improved solubility.

Within the context of the present invention, alkyl, alkenyl, alkynyl, alkoxy, aryl, arylene and N-heterocyclyl have the following meanings, unless otherwise specified:

Alkyl per se, and “alk” in alkoxy are a linear or branched alkyl radical having as a rule from 1 to 6, preferably from 1 to 4, particularly preferably from 1 to 3, carbon atoms, by way of example and preferably methyl, ethyl, n-propyl, isopropyl, tert-butyl, n-pentyl and n-hexyl.

Alkenyl is an alkyl radical having at least 2 carbon atoms and as a rule 1, 2 or 3 double bonds, for example and preferably ethenyl, n-propenyl and methylethenyl.

Alkynyl is an alkyl radical having at least 2 carbon atoms and as a rule 1, 2 or 3 triple bonds, for example and preferably ethynyl and propynyl.

Alkoxy is, by way of example and preferably, methoxy, ethoxy, n-propoxy, isopropoxy, tert-butoxy, n-pentoxy and n-hexoxy.

Aryl is a monocyclic to tricyclic aromatic, carbocyclic radical having as a rule from 6 to 14 carbon atoms, by way of example and preferably phenyl, naphthyl and phenanthrenyl. Arylene is a bivalent aryl radical, by way of example and preferably phenylene, naphthylene and phenanthrenylene.

N-Heterocyclyl is a monocyclic or polycyclic, preferably monocyclic or bicyclic, nonaromatic heterocyclic radical having as a rule from 4 to 10, preferably from 5 to 8, ring atoms and at least one N heteroatom, and in all up to 3, preferably up to 2, heteroatoms and/or hetero groups from the series N, O, S, SO and SO₂. The N-heterocyclyl radicals can be saturated or partially unsaturated. Preference is given to 5- to 8-membered, monocyclic, saturated N-heterocyclyl radicals having a total of up to two heteroatoms from the series O, N and S, such as, by way of example and preferably, pyrrolidin-2-yl, pyrrolidin-3-yl, pyrrolinyl, piperidinyl, morpholinyl and perhydroazepinyl, in particular pyrrolidin-2-yl and piperidin-4-yl.

The compounds which are not isotope-labeled already constitute one isotope coding. For the further isotope coding, the compounds according to the invention are preferably isotope-labeled with at least one carbon atom of the isotope ¹³C, in particular from four to 15 ¹³C atoms. The disadvantageous isotope effect which is observed in LC when using ¹H/²D affinity tags is markedly reduced by the ¹²C/¹³C isotope coding. Alternatively or additionally, it is also possible to use the isotopes ²D, ¹⁵N, ¹⁷O, ¹⁸O and/or ³⁴S for the labeling.

In one particular embodiment of the invention, ¹³C-labeled compounds are additionally isotope-labeled with at least one nitrogen atom of the isotope ¹⁵N, preferably from one to three ¹⁵N atoms, in particular one ¹⁵N atom.

The isotope labelings are usually performed in L and/or PRG, in particular in L, and, in the case of compounds of the formula (II), in CH₂ groups, B¹ and/or X¹.

The acid-cleavable group S is preferably arranged at the PRG-terminal end of the linker L and the spacer group Y directly bonded to the protein-reactive group PRG, for example in the form of compounds of the formula (II).

The degree of the acid-cleavability of the acid-cleavable group S depends on the structure of the given linker L and can be modulated and consequently adapted to the demands of the particular use of the compounds. If, for example, acid-cleavability under mild conditions is desired, compounds of the formula (II) in which B¹ is an amine group NRR′, in which R is not hydrogen, either, and in which, in particular, R and R′ are both alkyl or, together with N, are N-heterocyclyl, for example, and preferably, the amine groups N(CH₃)CH₂, pyrrolidin-2-yl and piperidin-4-yl, have proved to be particularly advantageous. Such mild conditions are provided, for example, by dilute trifluoroacetic acid, e.g. when the trifluoroacetic acid is diluted down to a content of less than 50% by vol., in particular less than 20% by vol., in a mixture of acetonitrile and water in a volume ratio of 1 to 1.

The compounds according to the invention can be prepared, for example, by initially reacting a group
H—B¹—X¹—(CH₂)ₙ—[X²—(CH₂)ₘ]ₓ—X³—(CH₂)ₚ—X⁴—V (III)
in which V═H or OH, preferably H,
in accordance with the coupling methods which are customary in peptide chemistry, with an amino acid derivative which is protected at the N-terminal amino group to give a conjugate
   SG-NH—CH(SK)—CO—B¹—X¹—(CH₂)ₙ—[X²—(CH₂)ₘ]ₓ—X³—(CH₂)ₚ—X⁴—V (IV)
   in which SG is a protecting group which is customary in peptide chemistry, preferably Boc, and in which SK is the residue of an amino acid side chain in the D or L configuration or in racemic form.

Subsequently, (IV) can be reacted with the derivative of a protein-reactive group or the activated precursor of a protein-reactive group of the formula
U—PRG (V)
in which U is a group which enables PRG to be linked to X⁴ by, for example, the group becoming a leaving group together with the residue V in (IV). Examples of such groups are activated esters such as N-hydroxysuccinimide esters or chlorides or groups from which a leaving group can be generated during the coupling.

In a further step, the amino-protecting group SG is then detached, resulting in a conjugate of the formula (VI)
H₂N—CH(SK)—CO—B¹—X¹—(CH₂)ₙ—[X²—(CH₂)ₘ]ₓ—X³—(CH₂)ₚ—X⁴—PRG (VI)

In parallel with this, the affinity ligand A-OH, or an activated form thereof, for example an activated ester or an acid chloride, is reacted, under suitable coupling conditions, with a compound 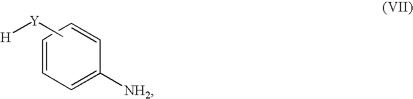
which can optionally also carry a protecting group, to give the compound

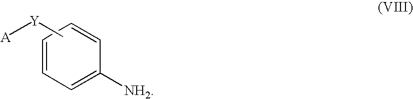

The compound (VIII) is then, where appropriate after prior elimination of an optionally introduced protecting group, converted, using activated carbonic acid derivatives such as thiophosgene or thiocarbonylbisimidazole, into a corresponding isothiocyanate and then coupled with (VI) to give the thiourea (IX).

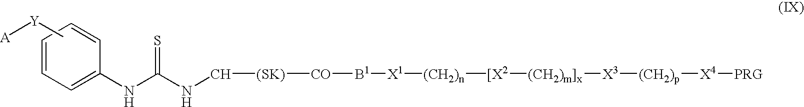

In the last step, any protecting groups which may possibly still be present can optionally be eliminated in order, in this way, to obtain conjugates of the formula (II).
A-S—B¹—X¹—(CH₂)ₙ—[X²—(CH₂)ₘ]—X³—(CH₂)ₚ—X⁴—PRG (II)

In a preferred embodiment of the preparation process, (IV) comprises a compound of the formula
Boc-NH—CH(SK)—CO—NH—(CH₂)₃—O—(CH₂)₂—O—(CH₂)₃—NH₂ (X)
which reacts with a compound of the formulae (XI a-c) 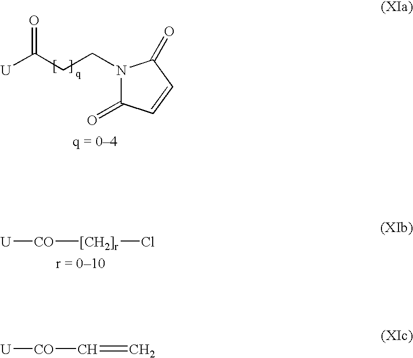
and, after the Boc protecting group has been detached, the compound which has been formed is reacted, in the presence of ethyldiisopropylamine or another suitable base, with an isothiocyanate which has been generated from compound (VIII), for example the isothiocyanate 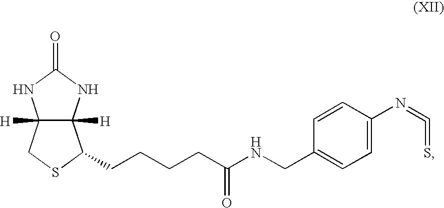
to give the target compounds of the formula (II).

In all the reaction steps, it is possible to use protecting groups which can be eliminated reversibly, as are customary in peptide chemistry. A protecting group SG can be retained or be detached at the same time as the Boc protecting group or in a separate step. Examples of suitable protecting groups are the Boc protecting group, which can be cleaved using trifluoroacetic acid, or the Fmoc protecting group, which can be cleaved using piperidine or morpholine. Other suitable protecting groups, and the appropriate methods for introducing and eliminating them, have been described, for example, in (a) Jakubke/Jeschkeit: Aminosäuren, Peptide, Proteine [Amino Acids, Peptides and Proteins]; Verlag Chemie 1982 or (b) Houben-Weyl, Methoden der Organischen Chemie [Methods of organic chemistry], Georg Thieme Verlag Stuttgart, fourth edition; volumes 15.1 and 15.2, edited by E. Wünsch.

The affinity tags according to the invention can also optionally be constructed in the reverse sequence, with standard methods ((a) Jakubke/Jeschkeit: Aminosäuren, Peptide, Proteine [Amino Acids, Peptides and Proteins]; Verlag Chemie 1982, (b) Houben-Weyl, Methoden der Organischen Chemie [Methods of organic chemistry], Georg Thieme Verlag Stuttgart, fourth edition; volumes 15.1 and 15.2, edited by E. W\ddot{u}nsch) initially being used to introduce a suitable additional protecting group SG′, preferably the Fmoc protecting group, into a derivative of the formula
SG-NH—CH(SK)—CO—B¹—X¹—(CH₂)ₙ—[X²—(CH₂)ₘ]ₓ—X³—(CH₂)ₚ—X⁴—V (IV)
thereby giving rise to
SG-NH—CH(SK)—CO—B¹—X¹—(CH₂)ₙ—[X²—(CH₂)ₘ]ₓ—X³—(CH₂)ₚ—X⁴—SG′ (XIII).

In the next step, the N-terminal protecting group SG is detached and the resulting derivative (XIII) is then reacted with the isothiocyanate, such as (XII) which is generated from (VII) to give compounds of the formula
A-S—B¹—X¹—(CH₂)ₙ—[X²—(CH₂)ₘ]ₓ—X³—(CH₂)ₚ—X⁴—SG′ (XIV).

After the protecting group SG′, for example the Fmoc protecting group, has been detached, for example using piperidine in the case of Fmoc, the resulting compound is coupled, in the last step, to the derivative of a protein-reactive group or the activated precursor of a protein-reactive group
U—PRG (V)
thereby giving rise to the conjugate
A-S—B¹—X¹—(CH₂)ₙ—[X²—(CH₂)ₘ]ₓ—X³—(CH₂)ₚ—X⁴—PRG (II).

The reactions can be carried out under different conditions of pressure and temperature, usually at from 0.5 to 2 bar, preferably under normal pressure, i.e. at about 1 bar, and at from −30 to +100° C, preferably at from −10 to +80° C, in particular at from 0 to 30° C. The reactions are carried out in suitable solvents such as dimethylformamide (DMF), tetrahydrofuran (THF), dichloromethane, chloroform, C₁-C₄-alcohols, acetonitrile, dioxane or water or in mixtures of these solvents. As a rule, preference is given to reactions in DMF, dichloromethane, THF, dioxane/water or THF/dichloromethane at room temperature (RT), i.e. about 20° C., or while cooling with ice, and under normal pressure.

### EXAMPLES

Abbreviations Employed:

Boc—tert-butoxycarbonyl
DIEA—diisopropylethylamine (Hünig's base)
DMAP—dimethylaminopyridine
DMF—dimethylformamide
DMSO—dimethyl sulfoxide
EDCI—N-(3-dimethylaminopropyl)-N′-ethylcarbodiimide, ×HCl
EI—electron impact ionization
ESI—electrospray ionization
Fmoc—fluorenyl-9-methoxycarbonyl
HOBT—1-hydroxy-1H-benzotriazole
HPLC—high-performance liquid chromatography
MALDI—matrix-assisted laser desorption ionization
MS—mass spectroscopy
MTBE—methyl tert-butyl ether
RP—reverse phase
RT—room temperature
TCEP—tris(carboxyethyl)phosphine
TFA—trifluoroacetic acid
THF—tetrahydrofuran
TLC—thin layer chromatography
(v/v)—concentration given in volume per volume
(w/v)—concentration given in mass per volume
aq.—aqueous

Unless otherwise expressly indicated, the composition of solvent and eluent mixtures is given by specifying the components, in each case separated by “/”, followed by the relative parts by volume. Thus, for example, “acetonitrile/water 10/1” denotes a mixture of acetonitrile and water in a volume ratio of 10 to 1.

Eluents which are preferably employed (referred to by specifying¹⁾ etc.):
¹⁾ acetonitrile/water 10/1
²⁾ acetonitrile/water 20/1
³⁾ dichloromethane/methanol 97.5/2.5
⁴⁾ acetonitrile/water/glacial acetic acid 10/1/0.1
⁵⁾ acetonitrile/water/glacial acetic acid 5/1/0.2
⁶⁾ acetonitrile/water/glacial acetic acid 10/3/1.5
⁷⁾ dichloromethane/methanol/aq. ammonia (17%) 15/2/0.2

In order to prepare exemplary affinity tags, as described below, the biotin derivatives of the starting compound series 1 and the intermediates of the starting compound series 2 were prepared first of all. These starting compounds were then reacted to give the corresponding affinity tags.
Starting Compound Series 1: Biotin Derivatives

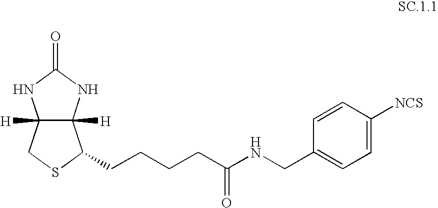

1 g (4.09 mmol) of biotin, 500 mg (4.09 mmol) of 4-aminobenzylamine, as well as 830 mg (6.14 mmol) of 1-hydroxy-1H-benzotriazole, 942 mg (4.91 mmol) of N-(3-dimethylaminopropyl)-N′-ethylcarbodiimide hydrochloride and 1587 mg of ethyldiisopropylamine were together added to 40 ml of DMF. The mixture was stirred overnight at room temperature and then concentrated and the residue was purified by flash chromatography on silica gel (elution mixture: dichloromethane/methanol/aq. ammonia (17%) 15/3/0.3). The appropriate fractions were combined and the solvent was evaporated off in vacuo. The residue was stirred up with diethyl ether and filtered off with suction. 1097 mg (77%) of the intermediate were obtained [TLC: dichloromethane/methanol/aq. ammonia (17%) 15/4/0.5: R_{f}=0.58] [ESI-MS: m/e=349 (M+H)⁺].

600 mg (1.72 mmol) of this intermediate were dissolved in 40 ml of dioxane/water 1/1, after which 298 mg (2.58 mmol) of thiophosgene and 890 mg of ethyldiisopropylamine were added. The mixture was stirred at room temperature for 10 min and concentrated. The target product SC.1.1. was precipitated with diethyl ether from dichloromethane/methanol. Yield: 616 mg (92%) [TLC: R_{f}=0.56⁵⁾] [ESI-MS: m/e=391 (M+H)⁺].

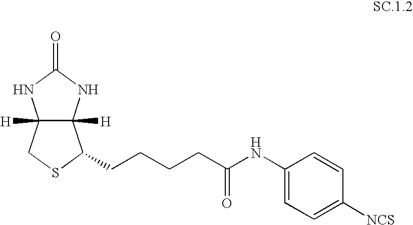

Mono-Fmoc-protected p-phenylenediamine was prepared using standard methods as are described, for example, in Houben Weyl; Methoden der Organischen Chemie [Methods of Organic Chemistry]; fourth edition; volume XV part 1 and 2; Georg Thieme Verlag Stuttgart 1974, or in Hans-Dieter Jakubke and Hans Jeschkeit: Aminosäuren, Peptide, Proteine [Amino Acids, Peptides and Proteins]; Verlag Chemie, Weinheim, 1982.

200 mg (0.82 mmol) of biotin were taken up in 10 ml of dichloromethane, after which 974 mg (8.2 mmol) of thionyl chloride were added. After the mixture had been stirred for 1 h, it was concentrated and the residue was subsequently distilled twice with dichloromethane.

The resulting acid chloride (0.81 mmol) was taken up in 30 ml of dichloromethane, after which 387 mg (4.9 mmol) of pyridine and 242 mg (0.55 mmol) of mono-Fmoc-protected p-phenylenediamine were added. The mixture was stirred at RT for 2 days and the product which had precipitated out was filtered off. This resulted in 300 mg (99%) of the intermediate, which was used in the next reaction step without any further purification [TLC: R_{f}=0.5¹⁾].

The crude product was taken up in 5 ml of DMF after which 500 μl of piperidine were added. After the mixture had been stirred at room temperature for 15 min, it was concentrated and the residue was purified by flash chromatography on silica gel (elution mixture⁷⁾. The appropriate fractions were combined, the solvent was removed and the residue was dried in vacuo. 69 mg (39%) of the deprotected intermediate were obtained.

65 mg (0.19 mmol) of this intermediate were dissolved in 10 ml of dioxane/water 1/1 and 33 mg (0.29 mmol) of thiophosgene and 100 mg of ethyldiisopropylamine were added to this solution. The mixture was stirred at room temperature for 10 min and concentrated. The target product SC.1.2 was precipitated with diethyl ether from dichloromethane/methanol. Yield: 65 mg (89%) [TLC: R_{f}=0.43¹⁾] [ESI-MS: m/e=377 (M+H)⁺].

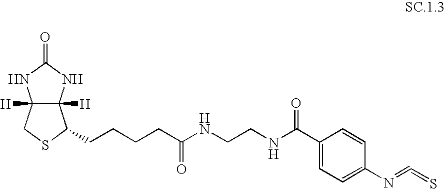

500 mg (3.65 mmol) of 4-aminobenzoic acid were taken up in 20 ml of DMF, after which 872 mg (2.73 mmol) of the hydrochloride of the mono-Fmoc-protected ethylenediamine, as well as 739 mg (5.47 mmol) of 1-hydroxy-1H-benzotriazole and 839 mg (4.38 mmol) of N-(3-dimethylaminopropyl)-N′-ethylcarbodiimide hydrochloride, were added. The mixture was stirred at room temperature for 2 h, after which it was concentrated and the residue was taken up in 200 ml of dichloromethane. This solution was then extracted by being shaken three times with 200 ml of a solution of sodium hydrogen carbonate. The organic phase was concentrated and the residue was purified by flash chromatography on silica gel (eluent: acetonitrile). The appropriate fractions were combined and the solvent was evaporated off in vacuo; the residue was then dried. This resulted in 639 mg (59%) of the intermediate [TLC: R_{f}=0.68²⁾].

400 mg (1 mmol) of the intermediate were taken up in 10 ml of DMF and 500 μl of piperidine were added to this solution. After the mixture had been stirred at room temperature for 15 min, it was concentrated and the residue was purified by flash chromatography on silica gel (elution mixture: dichloromethane/methano/aq. ammonia (17%) 15/4/0.5). The appropriate fractions were combined, the solvent was removed and the residue was dried in vacuo. 147 mg (82%) of the deprotected intermediate were obtained [TLC: acetonitrile/water/glacial acetic acid 5/1/0.2: R_{f}=0.18].

191 mg (0.78 mmol) of biotin were taken up in 10 ml of DMF, after which 158 mg (1.17 mmol) of 1-hydroxy-1H-benzotriazole and 180 mg (0.94 mmol) of N-(3-dimethylaminopropyl)-N′-ethylcarbodiimide hydrochloride were added. The mixture was stirred at RT for 10 min and 303 mg of ethyldiisopropylamine and 140 mg (0.78 mmol) of the deprotected intermediate were then added. The mixture was then once again stirred at RT for 6 h, after which it was concentrated and the crude product was precipitated with diethyl ether from dichloromethane. The residue was separated off and purified by flash chromatography on silica gel (elution mixture: dichloromethane/methanol/aq. ammonia (17%) 15/3/0.3). The appropriate fractions were combined and the solvent was evaporated off in vacuo, after which the residue was dried. 222 mg (70%) of the intermediate were obtained [TLC: dichloromethane/methanol/aq. ammonia (17%) 15/4/0.5: R_{f}=0.47].

200 mg (0.54 mmol) of this intermediate were dissolved in 15 ml of dioxane/water 1/1, after which 94 mg (0.81 mmol) of thiophosgene and 210 mg of ethyldiisopropylamine were added. The mixture was stirred at room temperature for 15 min and then concentrated. The target product was precipitated with diethyl ether from dichloromethane/methanol. Yield: 230 mg (95%/o) [TLC: acetonitrile/water/glacial acetic acid 5/1/0.2: R_{f}=0.44] [ESI-MS: m/e=448 (M+H)⁺].
Starting Compound Series 2

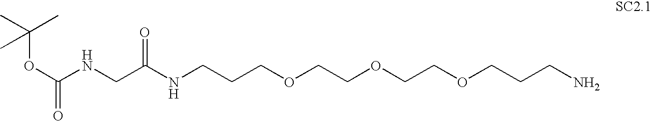

913.2 mg (4.54 mmol) of Boc-glycine N-carboxyanhydride and 100 mg of 4-dimethylaminopyridine were added to a solution of 4,7,10-trioxa-1,13-tridecane-diamine (1 g, 4.54 mmol) in 30 ml of dichloromethane and the mixture was stirred at RT for 16 h. It was then concentrated at reduced pressure, after which the residue was taken up in dichloromethane and this solution was washed with a little water. The organic phase was then dried over sodium sulfate and concentrated and the residue was purified by flash chromatography (eluent: dichloromethane/methanol/aq. ammonia (17%) 15/3/0.3). The appropriate fractions were collected, the solvent was evaporated off and the residue was dried under high vacuum. 333 mg (20%) of an oil were obtained [TLC: R_{f}=0.26⁵⁾].

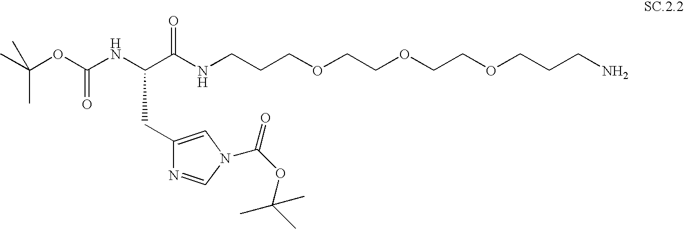

400 mg (0.88 mmol) of bis(tert-butoxycarbonyl)histidine N-hydroxysuccinimide ester were added to a solution of 4,7,10-trioxa-1,13-tridecanediamine (195 mg, 0.88 mmol) in 20 ml of dichloromethane and the mixture was stirred at RT for 30 min. The solvent was then removed in vacuo and the residue was purified by flash chromatography on silica gel (eluent: dichloromethane/methanol/17% ammonia 15/3/0.3). The appropriate fractions were combined and the solvent was evaporated in vacuo. The residue was taken up in dichloromethane and the solution was extracted by shaking with water. The organic phase was dried over sodium sulfate and then concentrated. 165 mg (34%) of an oil were obtained [TLC: R_{f}=0.31⁵⁾].

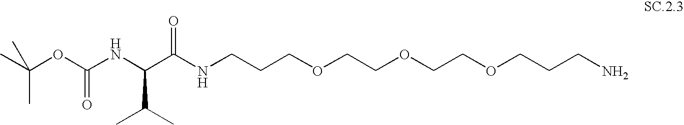

500 mg (2.3 mmol) of Boc-D-valine, 467 mg (3.45 mmol) of 1-hydroxy-1H-benzotriazole benzotriazole and 530 mg (2.76 mmol) of N-(3-dimethylaminopropyl)-N′-ethyl-carbodiimide hydrochloride were together added to 25 ml of DMF and the mixture was stirred at RT for 30 min. 507 mg (2.3 mmol) of 4,7,10-trioxa-1,13-tridecane-diamine were then added and the mixture was stirred at RT overnight. It was then concentrated and the residue was taken up in dichloromethane and this solution was extracted by being shaken twice with water. The organic phase was then concentrated and the residue was purified by flash chromatography on silica gel (eluent⁷⁾. The appropriate fractions were combined and the solvent was evaporated off in vacuo. The residue was taken up in dichloromethane and this solution was extracted by being shaken with water. The organic phase was dried over sodium sulfate and then concentrated. 220 mg (23%) of an oil were obtained (TLC: R_{f}=0.29⁵⁾].

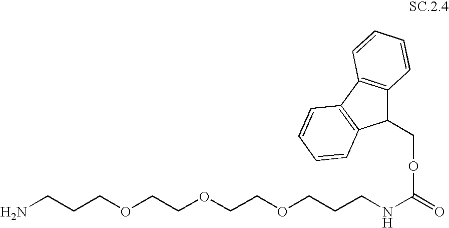

A solution of 9-fluoroenylmethyl chloroformate (23.5 g; 90.8 mmol) in tetrahydrofuran (500 ml) was slowly added dropwise, at −10° C., to the solution of 4,7,10-trioxa-1,13-tridecanediamine (20.0 g; 90.8 mmol) in 2-propanol (2000 ml). After 2 h, the mixture was concentrated under reduced pressure. The residue was taken up in dichloromethane (1000 ml) and this solution was washed twice with a saturated solution of sodium chloride (250 ml on each occasion). The organic phase was dried and concentrated. The residue was to a large extent homogeneous and free from the starting compound 4,7,10-trioxa-1,13-tridecanediamine. The yield of crude product was 36.5 g (91%) in the form of a syrup [TLC: dichloromethane/methanol 5/1: R_{f}=0.27] [ESI-MS: m/z 443.4 (M+H)⁺].

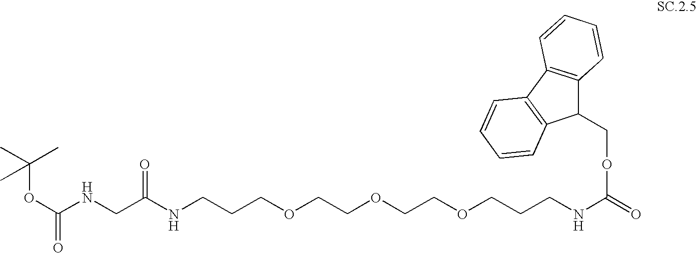

378 mg (2.2 mol) of Boc-glycine, as well as 364 mg (2.7 mmol) of 1-hydroxy-1H-benzotriazole and 413 mg (2.16 mmol) of N-(3-dimethylaminopropyl)-N′-ethyl-carbodiimide hydrochloride, were together added to 30 ml of DMF and the mixture was stirred at RT for 30 min. 1000 mg (1.8 mmol) of the compound SC.2.4 were then added and the mixture was stirred at RT for 2 h. It was then concentrated, after which the residue was taken up in dichloromethane and this solution was extracted by being shaken twice with water. The organic phase was then concentrated and the residue was purified by flash chromatography on silica gel (eluent²⁾). The appropriate fractions were combined and the solvent was evaporated off in vacuo. The residue was taken up in dichloromethane and this solution was extracted by being shaken with water. The organic phase was dried over sodium sulfate and then concentrated. 797 mg (76%) of an oil were obtained [TLC: R_{f}=0.64¹⁾].

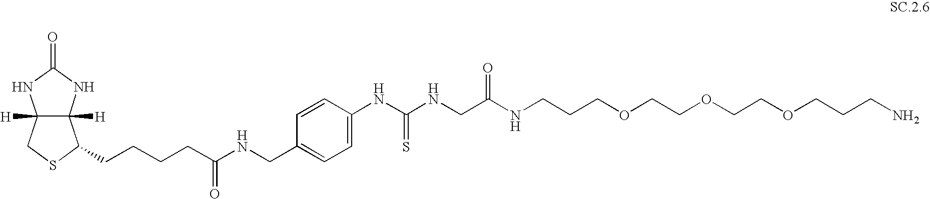

The Boc-protecting group was detached from the 790 mg (1.35 mmol) of the compound SC.2.5 in accordance with standard conditions using trifluoroacetic acid in dichloromethane (800 mg, 98%).

790 mg (1.32 mmol) of the deprotected intermediate were taken up in 30 ml of DMF, after which 514 mg (1.32 mmol) of the compound SC.1.1 and 511 mg of N,N-diisopropylethylamine were added and the mixture was stirred at RT for 16 h. The solvent was evaporated off and the residue was stirred up with 30 ml of water; the solid which remained was then filtered off. It was taken up in dichloromethane/methanol and the target compound was then precipitated with diethyl ether. 1077 mg (93%) were obtained after filtering and drying [TLC: R_{f}=0.4⁴⁾].

1070 mg (1.22 mmol) of this intermediate were taken up in 25 ml of DMF and 1 ml of piperidine was added. After the mixture had been stirred at room temperature for 45 min, it was concentrated and the residue was digested with dichloromethane. It was then filtered off and the filter residue was suspended in a mixture of 10 ml of dichloromethane and 10 ml of methanol. After 100 ml of diethyl ether had been added, the product precipitated completely and was filtered off and dried. 805 mg (99%) of the target product SC.2.6 were obtained [TLC: acetonitrile/water/glacial acetic acid 10/3/1.5 R_{f}=0.4].

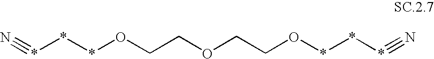

Diethylene glycol (3.18 g; 30 mmol) was added dropwise to a solution of aqueous potassium hydroxide (40%; 120 mg) and 1,4-dioxane (3.75 ml). 1,2,3-(¹³C)₃-acrylonitrile (3.36 g; 60 mmol) was then added dropwise to the solution while cooling with ice. After the solution had been warmed to room temperature, it was subsequently stirred for 16 h. The solution was diluted with dichloromethane (30 ml) and washed twice with a saturated solution of sodium chloride. The organic phase was dried and concentrated. The residue was purified by column chromatography (eluent: dichloromethane/methanol 50/1). Yield: 6.21 g (95%), consistency: syrup [TLC: dichloromethane/methanol 10/1: R_{f}=0.71; MS (ESI): m/z 241.1 (M+Na)⁺, 219.1 (M+H)⁺].

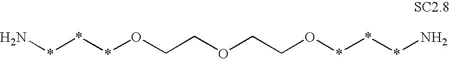

Raney nickel (2.5 g) was added to the solution of the compound SC.2.7 (5.0 g; 22.9 mmol) in methanol (115 ml) and a concentrated aqueous solution of ammonia (68 ml), and the mixture was hydrogenated with hydrogen for 5 h at 100° C. and 100 bar. After the mixture had cooled down to room temperature, the catalyst was filtered off with suction. The filtrate was concentrated. The residue was taken up three times in ethanol and concentrated. Yield: 3.84 g (74%), consistency: syrup [TLC: dichloromethane/methanol/ammonia 4/3/1: R_{f}=0.27] [MS (ESI): m/z 227.3 (M+H)⁺; ¹³C-NMR (100.6 MHz, CDCl₃): δ=69.48, 69.10 (¹³CH₂—O), 39.13, 38.77 (¹³CH₂—NH₂), 32.74, 32.38, 32.36, 32.01 (¹³CH₂—¹³CH₂—¹³CH₂)].

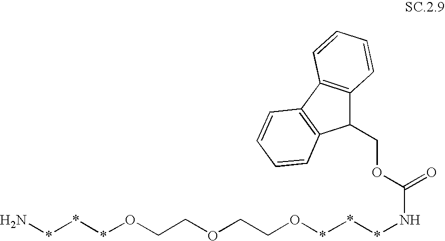

A solution of 9-fluorenylmethyl chloroformate (1.14 g; 4.42 mmol) in tetrahydrofuran (20 ml) was added, at −10° C., to the solution of the compound SC.2.8 (1.0 g; 4.42 mmol) in 2-propanol (50 ml). The mixture was warmed slowly to room temperature and stirred for 16 h. It was then concentrated. The residue was taken up in dichloromethane (50 ml) and this solution was washed twice with a saturated aqueous solution of sodium chloride, dried and concentrated. The residue was to a large extent homogeneous and was used in the following step without any further purification. Yield: 1.76 g (89%), consistency: syrup [TLC: dichloromethane/methanol 5/1: R_{f}0.27] [MS (ESI): m/z 449.4 (M+H)⁺].

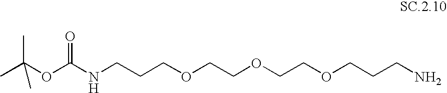

The Boc protecting group was introduced into 15 g (68 mmol) of 4,7,10-trioxa-1,13-tridecanediamine in accordance with standard conditions using di-tert-butyl dicarbonate in dichloromethane [TLC: R_{f}=0.34⁵⁾].

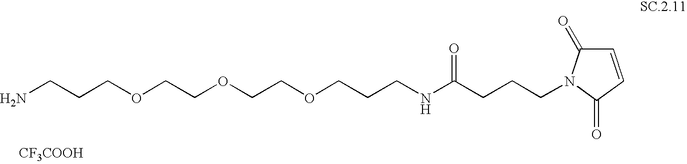

372 mg (2 mmol) of maleimidobutyric acid, and also 316 mg (2.34 mmol) of 1-hydroxy-1H-benzotriazole and 359 mg (1.87 mmol) of N-(3-dimethylamino-propyl)-N′-ethylcarbodiimide hydrochloride, were together added to 25 ml of DMF and the mixture was stirred at RT for 1 h. 500 mg (1.56 mmol) of the compound SC.2.10 and 403 mg of ethyldiisopropylamine were then added and the mixture was stirred at RT for a further 16 h. It was then concentrated and the residue was taken up in dichloromethane and this solution was extracted by being shaken four times with water. The organic phase was then dried over sodium sulfate and concentrated and the residue was precipitated with petroleum ether from dichloromethane. The supernatant was decanted off and the remaining oily residue dried under high vacuum. 577 mg (76%) of the desired product were obtained [TLC: R_{f}=0.48⁴⁾].

570 mg (1.17 mmol) of this intermediate were taken up in 10 ml of dichloromethane and 1 ml of TFA was added. After the mixture had been stirred at room temperature for 30 min, it was concentrated and the residue was precipitated with diethyl ether from dichloromethane. After filtration and drying, 90 mg (85%) of the desired product were obtained as trifluoroacetic acid salt [TLC: R_{f}=0.2⁵⁾].

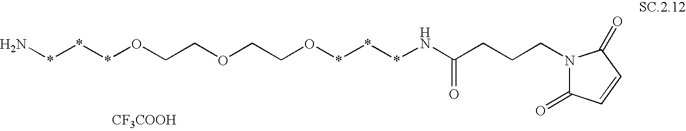

Preparation in analogy with Examples SC.2.10 and SC.2.11, proceeding from Example SC.2.8 [TLC: R_{f}=0.1⁵⁾]

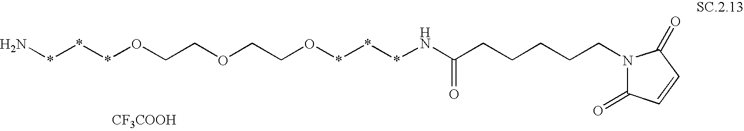

Preparation in analogy with Examples SC.2.10 and SC.2.11, proceeding from Example SC.2.8 [TLC: R_{f}=0.2⁵⁾].

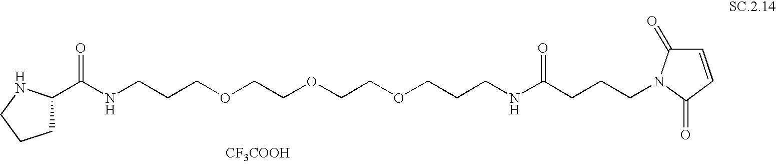

125 mg (0.4 mmol) of tert-butoxycarbonylproline N-hydroxysuccinimide ester and 155 mg of ethyldiisopropylamine were added to a solution of 200 mg (0.4 mmol) of the compound SC.2.11 in 15 ml of dimethylformamide and the mixture was stirred at RT for 30 min. The solvent was then removed in vacuo and the residue was purified by flash chromatography on silica gel (eluent: acetonitrile/water 20/1). The appropriate fractions were combined, the solvent was evaporated off in vacuo and the residue was dried. The Boc protecting group was then detached in accordance with standard conditions.

Yield: 75% over 2 steps [TLC: R_{f}=0.22⁵⁾].

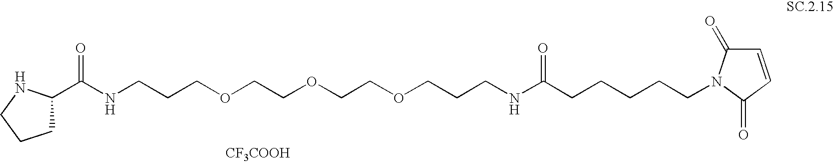

Preparation in analogy with SC.2.14.

Yield: 54% over 2 steps [TLC: R_{f}=0.23⁵⁾].

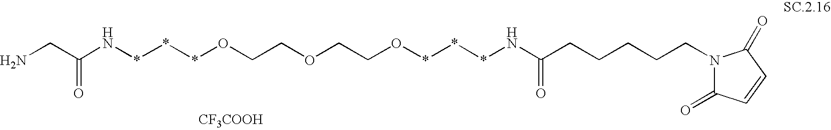

Preparation in analogy with Example SC.2.14, proceeding from SC.2.13.

Yield: 55% over 2 steps [TLC: R_{f}=0.1⁵⁾].

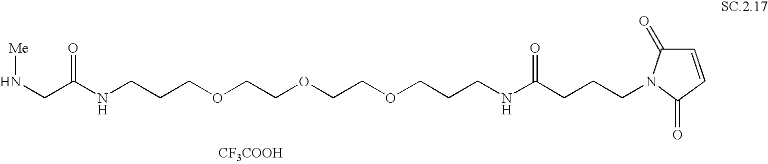

Prepared by linking Boc-sarcosine to compound SC.2.11 in the presence of EDCI/HOBT and then eliminating the Boc using standard conditions.

Yield: 56% over 2 steps [TLC: R_{f}=0.14⁵⁾].

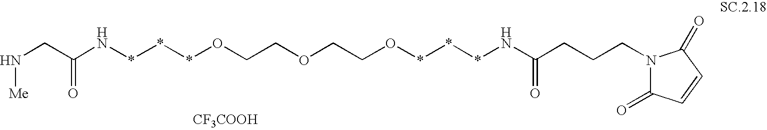

Preparation in analogy with Example SC.2.17, proceeding from Example SC.2.12.

Yield: 71% over 2 steps [TLC: R_{f}=0.1⁵⁾].

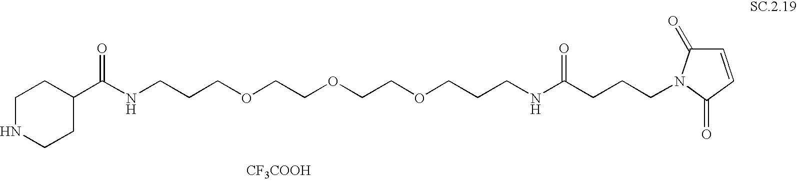

Prepared by linking Boc-piperidine-4-carboxylic acid to compound SC.2.11 in the presence of EDCI/HOBT and then eliminating the Boc using standard conditions.

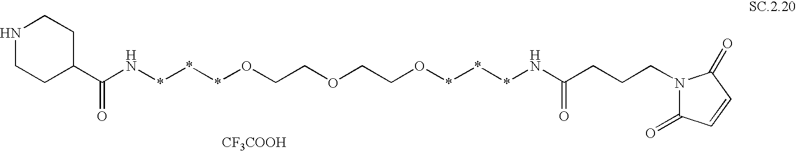

Preparation in analogy with Example SC.2.19, proceeding from Example SC.2.12.

Yield: 51% over 2 steps [TLC: R_{f}=0.08⁵⁾].

### Examples of Acid-Cleavable Affinity Tags

### Example 1

### Preparation Process (Variant A)

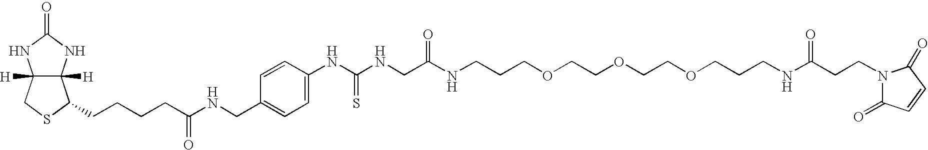

45 mg (265 μmol) of maleimidopropionic acid, as well as 54 mg (0.397 mmol) of 1-hydroxy-1H-benzotriazole and 61 mg (0.318 mmol) of N-(3-dimethylamino-propyl)-N′-ethylcarbodiimide hydrochloride, were together added to 10 ml of DMF and the mixture was stirred at RT for 2 h. 100 mg (0.265 mmol) of the compound SC.2.1 and 103 mg of ethyldiisopropylamine were then added and the mixture was stirred at RT for a further 2 h. It was then concentrated and the residue was taken up in dichloromethane; this solution was then extracted by being shaken four times with water. The organic phase was then dried over sodium sulfate and concentrated and the residue was precipitated with petroleum ether from dichloromethane. The supernatant was decanted off and the remaining oily residue was dried under high vacuum. 103 mg (74%) of the desired product were obtained [TLC: R_{f}=0.48⁴⁾].

100 mg (189 μmol) of this intermediate were taken up in 10 ml of dichloromethane and 1 ml of TFA was added. After the mixture had been stirred at room temperature for 30 min, it was concentrated and the residue was precipitated with diethyl ether from dichloromethane. After filtration and drying, 90 mg (85%) of the desired product were obtained as trifluoroacetic acid salt [TLC: R_{f}=0.58⁶⁾].

88 mg (158 μmol) of the deprotected intermediate and 62 mg (158 μmol) of the isothiocyanate SC.1.1 from starting compound series 1 were dissolved in 10 ml of DMF, after which 83 μl of ethyldiisopropylamine were added and the mixture was stirred at RT for 3 h. It was concentrated and the residue was stirred up with water and filtered off with suction. The residue was isolated and then taken up in 4 ml of dichloromethane/methanol 1/1 and 2 ml of DMF and precipitated with diethyl ether. The precipitate was filtered off with suction and, after drying under high vacuum, 110 mg (85%) of the target compound were obtained [TLC: R_{f=}0.5⁵⁾] [ESI-MS: m/e=819 (M+H)⁺].

### Example 2

### Preparation Process (Variant B)

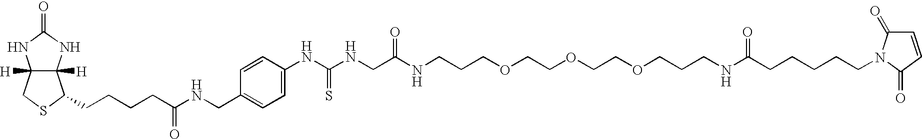

790 mg (1.35 mmol) of the compound SC.2.5 were taken up in 40 ml of dichloromethane and 8 ml of TFA were added. After the mixture had been stirred at room temperature for 1 h, it was concentrated and the residue was precipitated with diethyl ether from dichloromethane. The supernatant was decanted off and the resinous product was dried under high vacuum. 800 mg (99%) of the desired product were obtained as trifluoroacetic acid salt [TLC: R_{f}=0.3⁵⁾].

790 mg (1.32 mmol) of the deprotected intermediate were initially introduced in 30 ml of DMF, after which 515 mg (1.32 mmol) of the isothiocyanate SC.1.1 from the starting compound series 1 and 690 μl of ethyldiisopropylamine were added and the mixture was then stirred overnight at RT. It was concentrated and the residue was stirred up with 10 ml of water and filtered off with suction. The residue was isolated and then suspended in dichloromethane/methanol. 50 ml of diethyl ether were added and the precipitate was filtered off once again. After filtration and drying, 1077 mg (93%) of the desired compound were obtained [TLC: R_{f}=0.4⁴⁾).

1070 mg (1.22 mmol) of this intermediate were taken up in 25 ml of DMF and 1 ml of piperidine was added. After the mixture had been stirred at room temperature for 45 min, it was concentrated and the residue was digested with dichloromethane. It was then filtered off and the filter residue was suspended in a mixture of 10 ml of dichloromethane and 10 ml of methanol. After 100 ml of diethyl ether had been added, the product precipitated out completely and was filtered and dried. 805 mg (99%) of the % of the deprotected intermediate SC.2.6 were obtained [TLC: R_{f}=0.4⁶⁾].

9.5 mg (45 mmol) of maleimidocaproic acid, as well as 9.1 mg (0.067 mmol) of 1-hydroxy-1H-benzotriazole and 10.3 mg (0.054 mmol) of N-(3-dimethylamino-propyl)-N′-ethylcarbodiimide hydrochloride, were together added to 15 ml of DMF and the mixture was stirred at RT for 1.5 h. 30 mg (0.265 mmol) of the intermediate which was deprotected in the previous step (compound SC.2.6) and 24 μl of N,N-diisopropylethylamine were then added and the solution was stirred overnight at RT. The solvent was evaporated off and the residue was taken up in 3 ml of dichloromethane/methanol 1/1 and 15 ml of diethyl ether were then added. The precipitated crude product was purified by flash chromatography on silica gel (eluent: acetonitrile/water 10/1). The appropriate fractions were combined and the solvent was evaporated off in vacuo. The residue was dissolved in dioxane/water 1/1 and lyophilized. 6 mg (16%) of the target product were obtained [TLC: R_{f}=0.2¹⁾] [ESI-MS: m/e=861 (M+H)⁺].

Unless otherwise indicated, the following examples were prepared in an analogous manner to Example 1 (variant A) or Example 2 (variant B). In that which follows, the relevant variant is either mentioned or described.

### Example 3

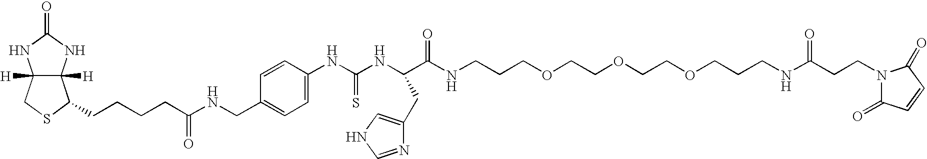

Starting compounds: SC.2.2, SC.1.1; variant A

Yield: 11% over 3 steps

R_{f}=0.65⁶⁾

[ESI-MS: m/e=899 (M+H)⁺]

The compound is completely soluble in water.

### Example 4

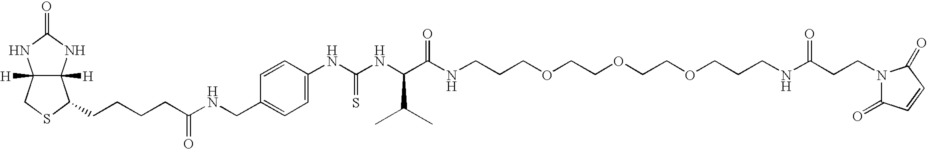

Starting compounds: SC.2.3, SC.1.1; variant A

Yield: 23% over 3 steps

R_{f}=0.67⁵⁾

[ESI-MS: m/e=861 (M+H)⁺]

### Example 5

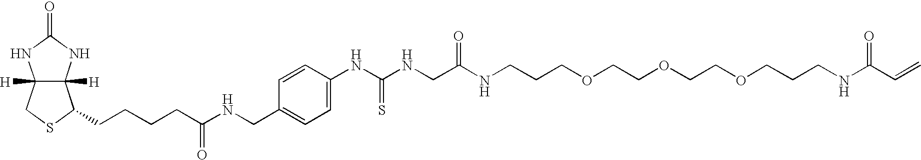

50 mg (75 μmol) of the compound SC.2.6 were-suspended in dichloromethane and 18 μl (225 μmol) of acryloyl chloride and 12 μl of pyridine were added in portions under argon. The mixture was then stirred at RT for 2 days. The solvent was evaporated off and the residue was purified by flash chromatography on silica gel (eluent¹⁾. The appropriate fractions were combined and the solvent was evaporated off in vacuo. The residue was dissolved in dioxane/water 1/1 and lyophilized. 12 mg (22%) of the target product were obtained [TLC: R_{f}=0.14¹⁾] [ESI-MS: m/e=722 (M+H)⁺].

### Example 6

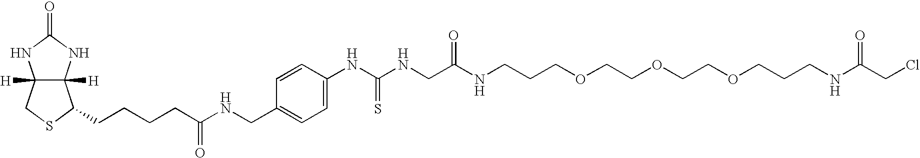

50 mg (75 μmol) of the compound SC.2.6 were suspended in 20 ml of dichloromethane, after which 24 μl (300 μmol) of chloroacetyl chloride and 12 μl of pyridine were added under argon. The mixture was then stirred at RT for 2 h. The solvent was evaporated off and the residue was treated with 5 ml of dichloromethane.

The remaining solid was isolated and purified by flash chromatography on silica gel (eluent¹⁾). The appropriate fractions were combined and the solvent was evaporated off in vacuo. The residue was taken up in dichloromethane/methanol and the target product was precipitated with diethyl ether. 12 mg (22%) of the target product were obtained [TLC: R_{f}=0.21¹⁾] [ESI-MS: m/e=744 (M+H)⁺].

### Example 7

### Preparation Process (Variant C)

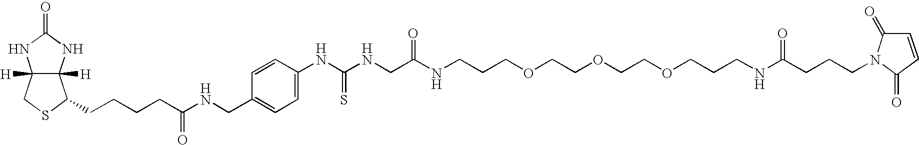

The compound SC.2.11 was reacted, as described in Example SC.2.5, with Boc-glycine. Yield: 61% [TLC: R_{f}=0.52⁴⁾]

The Boc protecting group was then detached using trifluoroacetic acid in dichloromethane. Yield: 98% [TLC: R_{f}=0.2⁵⁾]

This deprotected intermediate and 62 mg (158 μmol) of the isothiocyanate SC.1.1 from the starting compound series 1 were then reacted with each other, as described in Example 1, in order to obtain the target product. [TLC: R_{f}=0.4⁵⁾]

### Example 8

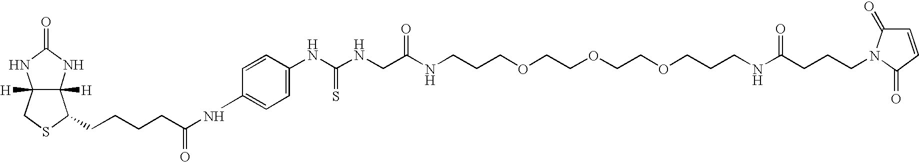

Starting compounds: SC.2.11; Boc-glycine; SC.1.2 variant C

[TLC: R_{f}=0.5⁵⁾]

### Example 9

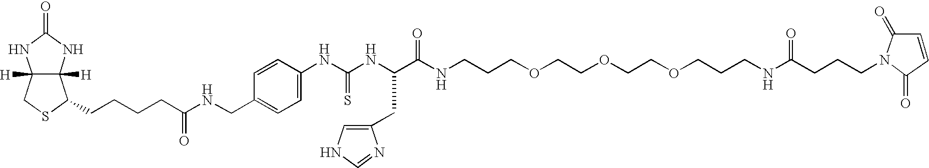

Starting compounds: SC.2.11; bis-Boc-histidine; SC.1.1 variant C

[TLC: R_{f}=0.35⁶⁾]

### Example 10

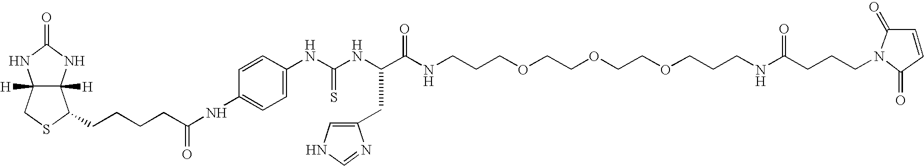

Starting compounds: SC.2.11; bis-Boc-histidine; SC.1.2 variant C

[TLC: R_{f}=0.35⁶⁾]

### Example 11

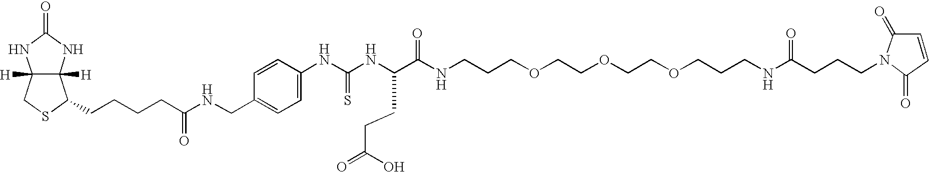

Starting compounds: SC.2.11; δtert-butyl Boc-glutamate; SC.1.1 variant C

[TLC: R_{f}=0.5⁵⁾]

### Example 12

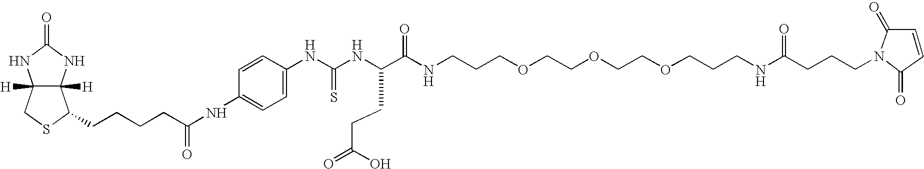

Starting compounds: SC.2.11; δ-tert-butyl Boc-glutamate; SC.1.2 variant C

[TLC: R_{f}=0.7⁶⁾]

### Example 13

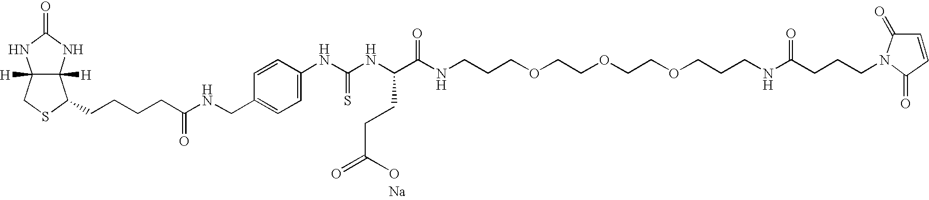

Starting compounds: SC.2.11; δ-tert-butyl Boc-glutamate; SC.1.1

Variant C and subsequent conversion into the sodium salt using 1 equivalent of an aqueous solution of sodium hydroxide.

Yield: 38% over 4 steps [TLC: R_{f}=0.4⁵⁾] [ESI-MS: m/e=905 (M+H)⁺].

### Example 14

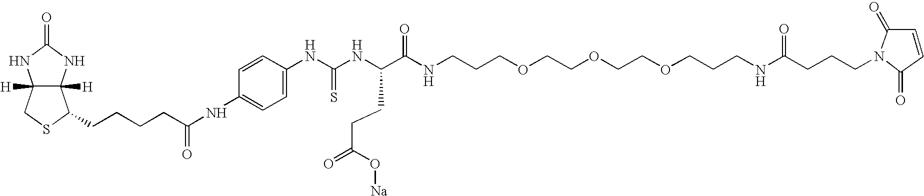

Starting compounds: SC.2.11; δ-tert-butyl Boc-glutamate; SC.1.2

Variant C and subsequent conversion into the sodium salt using 1 equivalent of an aqueous solution of sodium hydroxide.

Yield: 52% over 4 steps [TLC: R_{f}=0.5⁵⁾] [ESI-MS: m/e=891 (M+H)⁺].

### Example 15

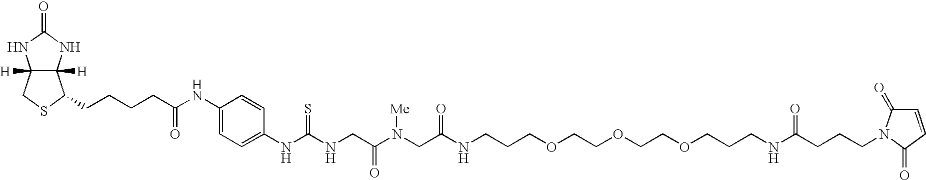

Starting compounds: SC.2.17; Boc-glycine N-carboxylic acid anhydride; SC.1.2

Variant C, with Boc-glycine N-carboxylic acid anhydride being used as the activated amino acid building block, instead of the activation by way of EDCI/HOBT.

Yield: 55% over 3 steps [TLC: R_{f}=0.45⁵⁾] [ESI-MS: m/e=890 (M+H)⁺].

### Example 16

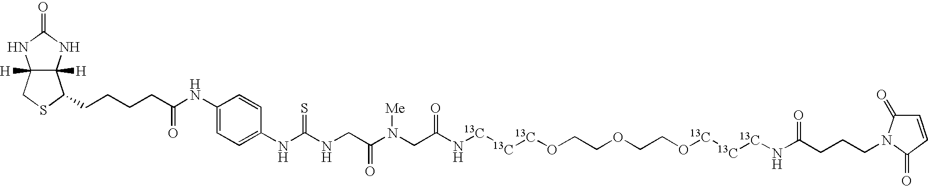

Starting compounds: SC.2.18; Boc-glycine; SC.1.2 variant C

Yield: 37% over 3 steps [TLC: R_{f}=0.4⁵⁾] [ESI-MS: m/e=896 (M+H)⁺].

### Example 17

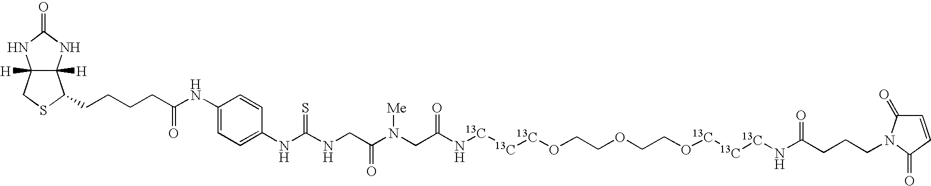

Starting compounds: SC.2.14; Boc-glycine N-carboxylic acid anhydride; SC.1.2

Variant C, with Boc-glycine N-carboxylic acid anhydride being used as the activated amino acid building block instead of the activation by way of EDCI/HOBT.

Yield: 47% over 3 steps [TLC: R_{f}=0.45⁵⁾] [ESI-MS: m/e=916 (M+H)⁺].

### Example 18

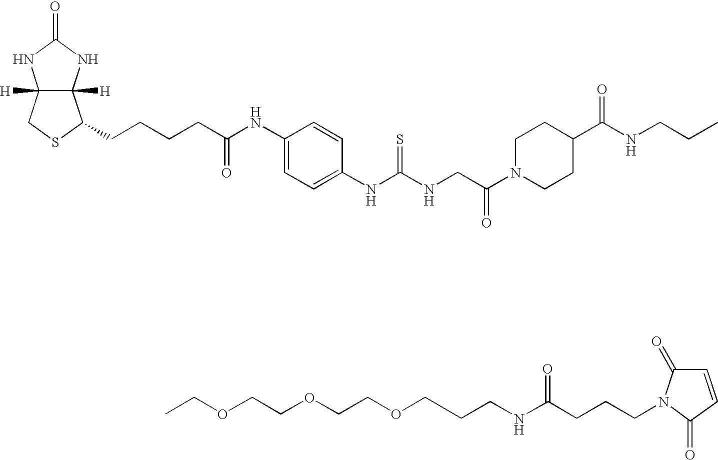

Starting compounds: SC.2.19; Boc-glycine; SC.1.2 variant C

Yield: 47% over 3 steps [TLC: R_{f}=0.5⁵⁾] [ESI-MS: m/e=930 (M+H)⁺].

### Example 19

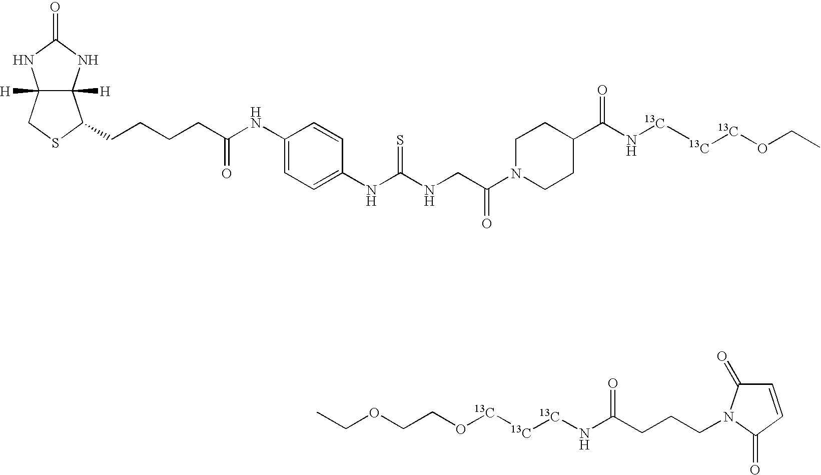

Starting compounds: SC.2.20; Boc-glycine; SC.1.2 variant C

Yield: 32% over 3 steps [TLC: R_{f}=0.5⁵⁾] [ESI-MS: m/e=936 (M+H)⁺].

### Example 20

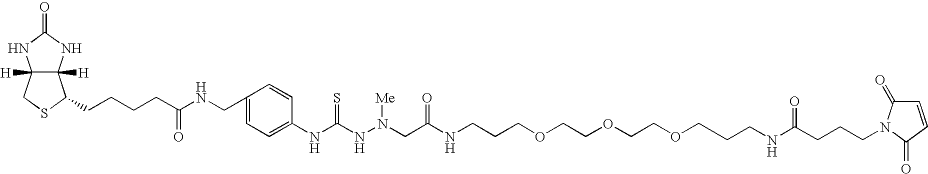

Starting compounds: SC.2.17; Boc-glycine N-carboxylic acid anhydride; SC.C.1

Variant C, with Boc-glycine N-carboxylic acid anhydride being used as the activated amino acid building block instead of the activation by way of EDCI/HOBT.

Yield: 55% over 3 steps [TLC: R_{f}=0.46⁵⁾] [ESI-MS: m/e=904 (M+H)⁺].

### Example 21

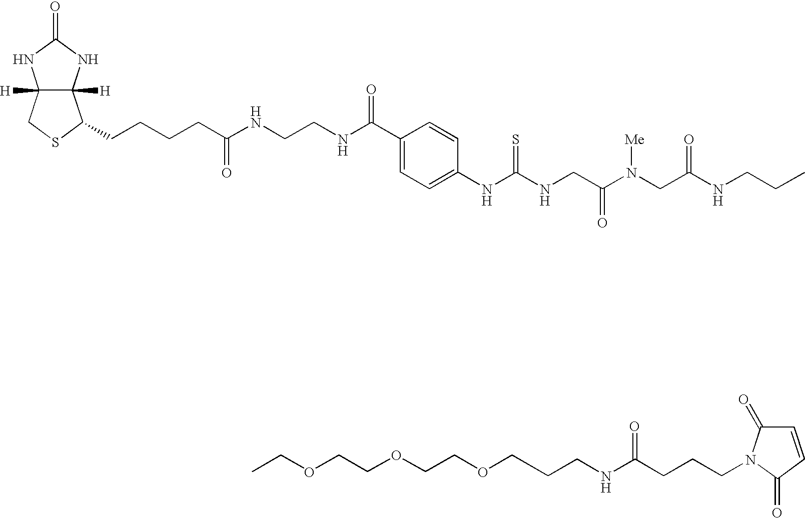

Starting compounds: SC.2.17; Boc-glycine N-carboxylic acid anhydride; SC.1.3

Variant C, with Boc-glycine N-carboxylic acid anhydride being used as the activated amino acid building block instead of the activation by way of EDCI/HOBT.

Yield: 38% over 3 steps [TLC: R_{f}=0.4⁵⁾] [ESI-MS: m/e=961 (M+H)⁺].

### Example 22

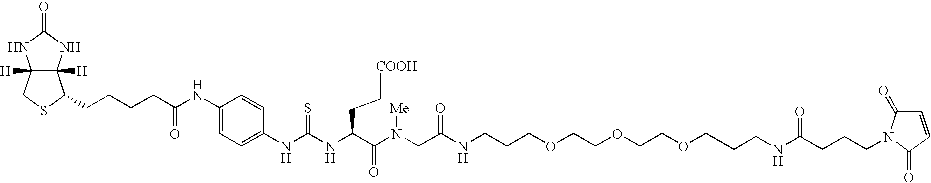

Starting compounds: SC.2.17; 6-tert-butyl Boc-glutamate; SC.1.2 variant C

Yield: 19% over 3 steps [TLC: R_{f}=0.46⁵⁾] [ESI-MS: m/e=962 (M+H)⁺].

### Example 23

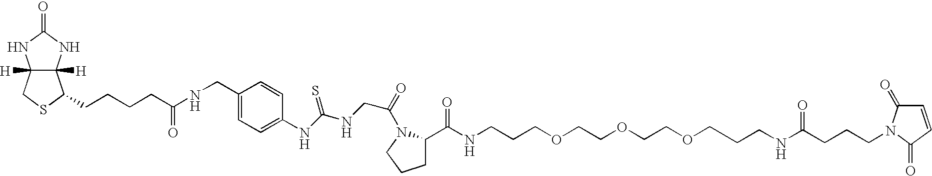

Starting compounds: SC.2.14; Boc-glycine N-carboxylic acid anhydride; SC.1.1

Variant C, with Boc-glycine N-carboxylic acid anhydride being used as the activated amino acid building block instead of the activation by way of EDCI/HOBT.

Yield: 47% over 3 steps [TLC: R_{f}=0.44⁵⁾] [ESI-MS: m/e=930 (M+H)⁺].

### Example 24

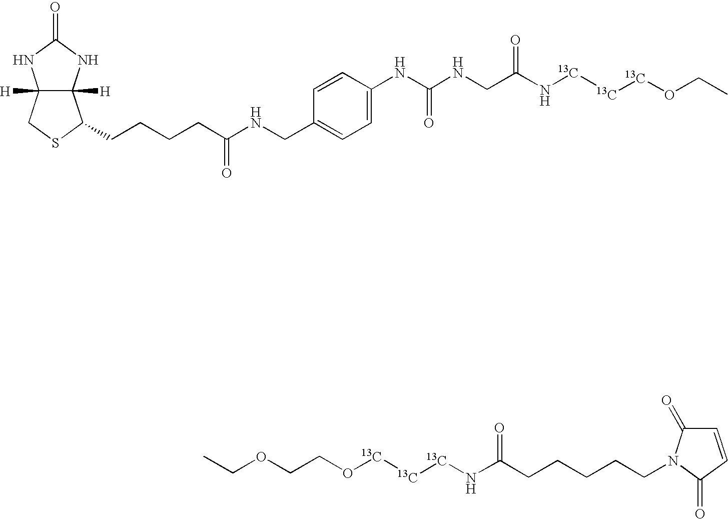

Starting compounds: SC.2.13; Boc-glycine; SC.1.1 variant C

Yield: 42% over 3 steps [TLC: R_{f}=0.57⁵⁾] [ESI-MS: m/e=867 (M+H)⁺].

### Example 25

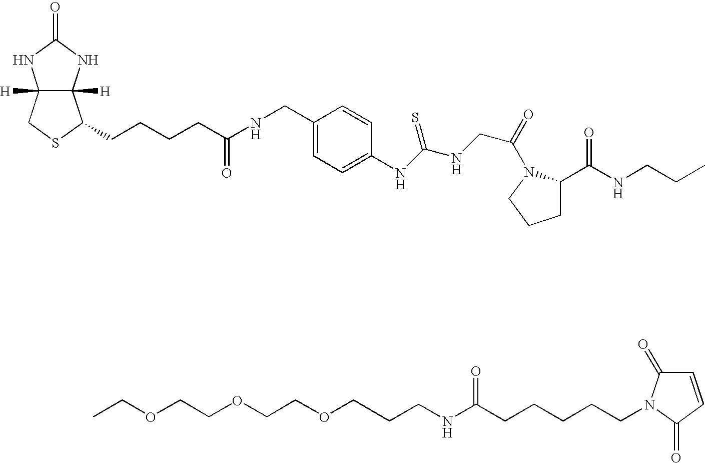

Starting compounds: SC.2.15; Boc-glycine N-carboxylic acid anhydride; SC.1.1

Variant C, with Boc-glycine N-carboxylic acid anhydride being used as the activated amino acid building block instead of the activation by way of EDCI/HOBT.

Yield: 37% over 3 steps [TLC: R_{f}=0.5⁵⁾] [ESI-MS: m/e=958 (M+H)⁺].

Investigations Involving Protein Analysis

Coupling the Affinity Tags to SDS-7 and Description of the Operational Procedure using Example 2

A mixture of seven proteins, which is also used as a size standard in gel electrophoresis (SDS-7 markers, Sigma-Aldrich GmbH, Taufkirchen) was used as the sample.

24 μg of the protein mixture were dissolved in 5 μl of buffer 1 and this solution was diluted with 135 μl of buffer 3. The proteins were denatured by heating at 100° C. for 3 minutes. In order to reduce the cysteines which were present, 3 μl of reducing solution were added and the mixture was incubated at 1 00° C. for 10 minutes. In order to react the free cysteines with the affinity tag, 5 μl of derivatizing solution were then added and the mixture was incubated at 37° C. for 90 minutes.

After the derivatization, 3 μl of trypsin solution were added. The proteins are then cleaved overnight (approx. 17 hours) at 37° C.

Buffer 1: 50 mM Tris-HCl, pH 8.3; 5 mM EDTA; 0.5% (w/v) SDS

Buffer 2: 10 mM NH₄acetate, pH 7

Buffer 3: 50 mM Tris-HCl, pH 8.3; 5 mM EDTA

Reducing solution: 50 mM TCEP in buffer 2

Derivatizing solution: 30 μg of affinity tag/μl in DMSO

Trypsin solution: 1 mg of trypsin (Promega GmbH, Mannheim)/ml in buffer 3

Affinity Purification of Derivatized Peptides

The affinity columns (monomeric avidin, Perbio Science Deutschland GmbH, Bonn), having a column volume of 200 μl, were prepared freshly prior to the purification and made ready by means of the following washing steps:
two column volumes of 2×PBS
four column volumes of 30% (v/v) acetonitrile/0.4% (v/v) trifluoroacetic acid
seven column volumes of 2×PBS
four column volumes of 2 mM biotin in 2×PBS
six column volumes of 100 mM glycine, pH 2.8
six column volumes of 2×PBS

Prior to loading, 30 μl of sample were diluted with 30 μl of 2×PBS, after which the diluted sample was loaded onto the column. After that, the following washing steps were carried out in order to remove the unbiotinylated peptides:
six column volumes of 2×PBS
six column volumes of PBS
six column volumes of 50 mM ammonium hydrogen carbonate/20% (v/v) methanol
one column volume of 0.3% (v/v) formic acid

The sample was eluted by means of the following steps:
three column volumes of 0.3% (v/v) formic acid
three column volumes of 30% (v/v) acetonitrile/0.4% (v/v) trifluoroacetic acid

The eluate was evaporated down to dryness and only dissolved once again shortly before carrying out the mass spectrometric analysis.

PBS: 10× stock solution, GibcoBRL, Cat. No. 14200-067

Mass-Spectrometric Analysis

An ion trap mass spectrometer (LCQdeka, ThermoFinnigan, San Jose) which was connected directly to a high pressure liquid chromatography appliance (LC-MS) was used for analyzing the peptides. A reversed-phase column (C₁₈ phase) was used as the separation column. The peptides were dissolved in Eluent A (0.025% (v/v) trifluoroacetic acid) and injected. They were eluted with a gradient of eluent B (0.025% (v/v) trifluoroacetic acid/84% (v/v) acetonitrile). The eluting peptides were recognized automatically by the acquisition software in the instrument and fragmented for identification. In this way, it was possible to determine the identities of the peptides unambiguously.

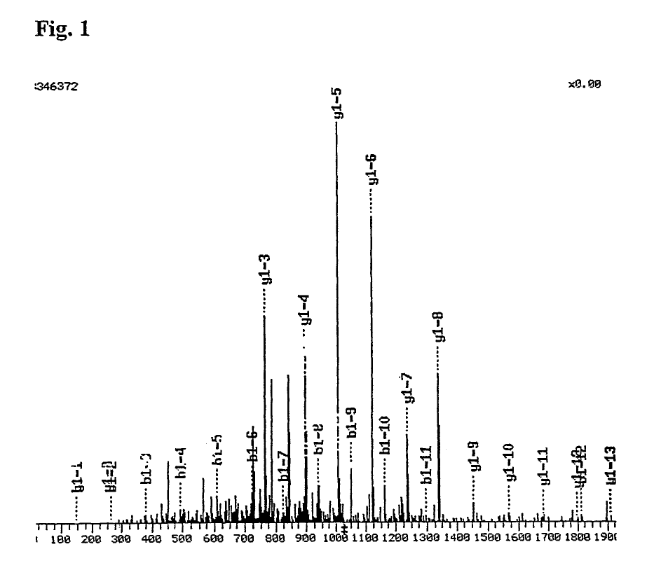

FIG. 1 shows an example of a fragment spectrum of a peptide from this analysis. The observed pattern identifies the peptide unambiguously as being the peptide having the sequence FLDDDLTDDIMCVK from lactalbumin, which was a constituent of the sample. The mass of the peptide, and its fragmentation, confirm that the affinity tag was cleaved by acid in the expected manner.

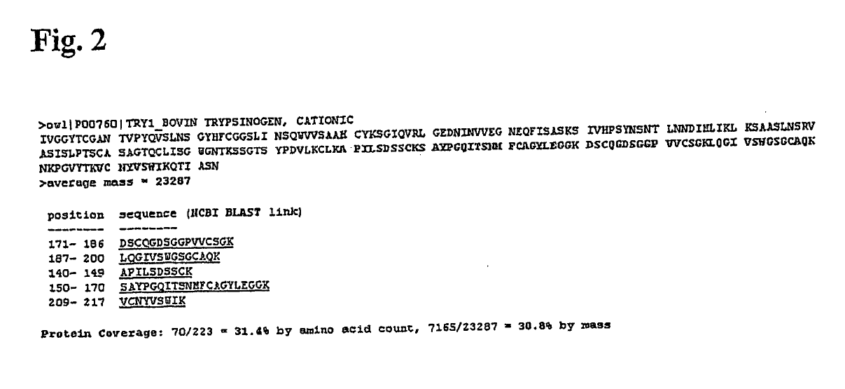

In all, 18 different peptides from the sample, all of which peptides carried the expected mass of the adduct with the acid-cleaved affinity tag residue in the same manner, were identified in one analysis. No cystein-containing peptide which was still carrying a complete affinity tag residue was identified. As an example, FIG. 2 shows the peptides which were identified from bovine trypsin.

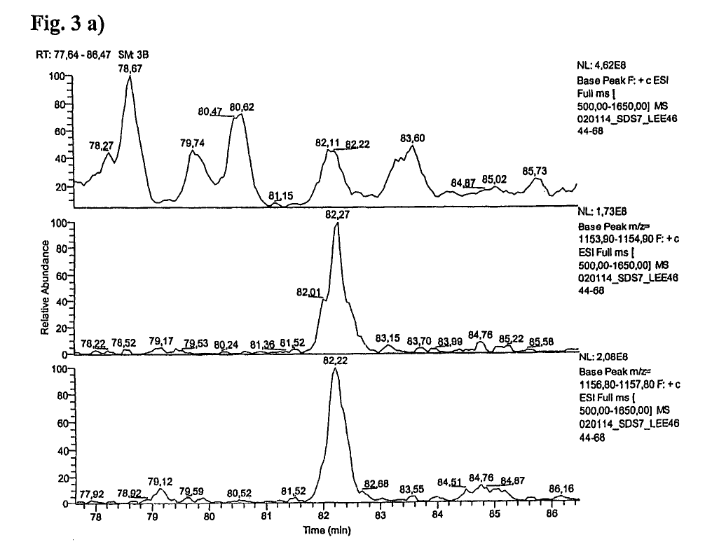

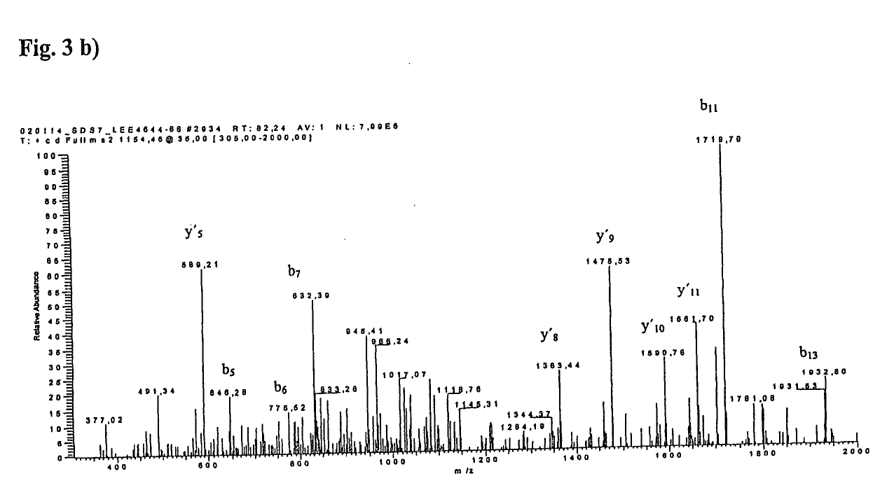

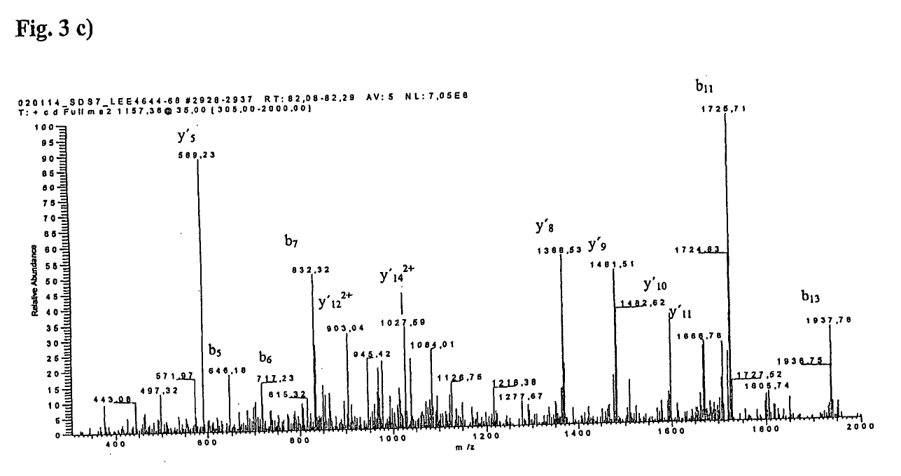

With the aid of Examples 15 and 16, FIG. 3 shows that the isotope-labeled affinity tags behave identically, as regards chromatographic and mass-spectrometric properties, independently of the degree of labeling. By way of example, FIG. 3a) shows the ion traces of the light and heavy variants of the labeled peptide LFTFHADICTLPDTEKD from bovine albumin. It is not possible to detect any difference in the retention time. FIGS. 3b) and c) show the fragment spectra of the doubly charged peptide ions. The two are identical apart from the shift of the cysteine-containing fragments by 6 Da due to the isotope labeling.

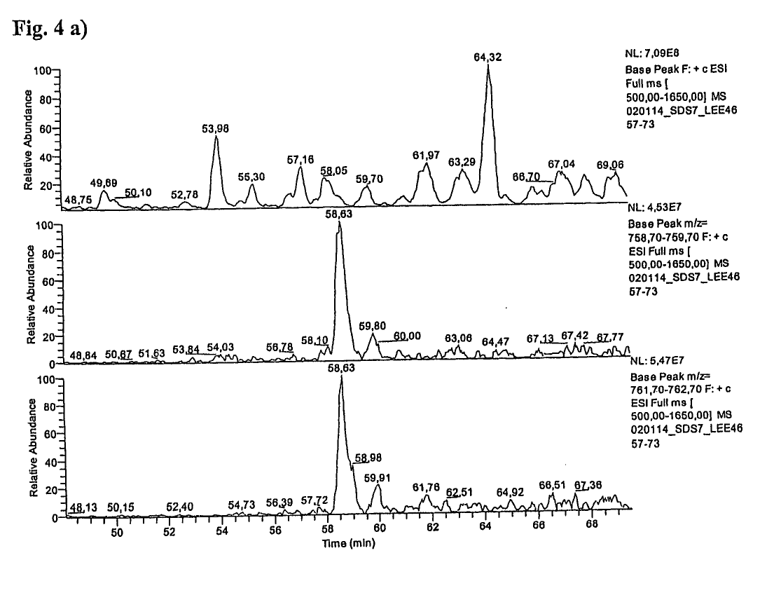

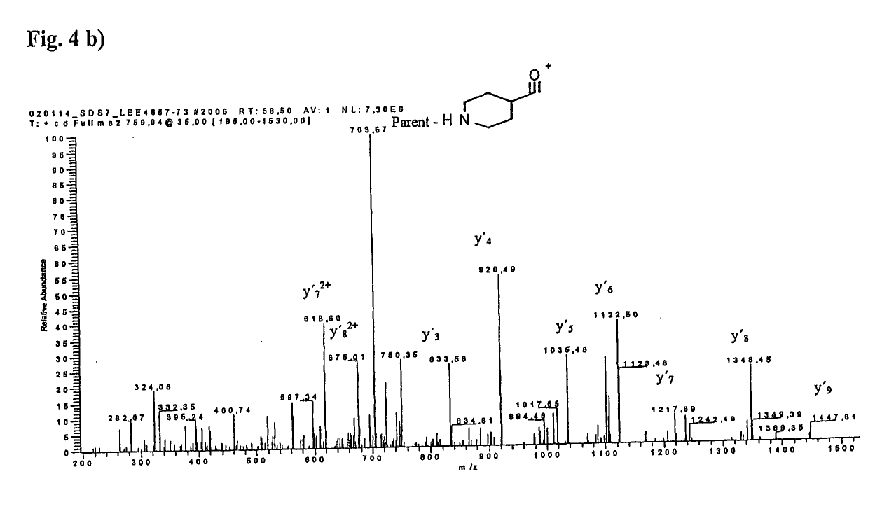

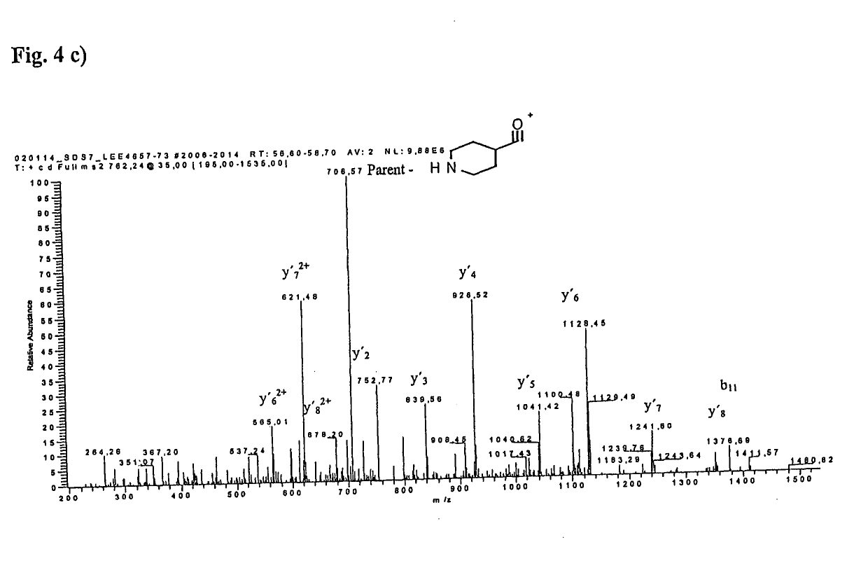

The same results are also obtained for other affinity tags and peptides, as FIG. 4 substantiates in the case of Examples 18 and 19. This figure relates to the bovine trypsin peptide APILSDSSCK. In this case, too, it is possible to observe precise coelution in the chromatography and identical behavior in connection with the fragmentation in the mass spectrometer.

FIG. 1: Fragment spectrum of a peptide derivatized with the compound from Example 2, following isolation using avidin, i.e. possessing an acid-cleaved affinity tag.

FIG. 2: Coverage of the bovine trypsin sequence which was achieved when using the compound from Example 2.

FIG. 3: MS analysis of a protein mixture following derivatization with Examples 15 and 16 in one single mixture. It can be seen from the ion chromatograms (FIG. 3a)) that the two variants elute at precisely the same time and appear with equal intensities. Apart from the expected shifts of 6 Da, the fragment spectra of the light (FIG. 3c)) and heavy (FIG. 3c)) variants of the labeled peptide LFTFHADICTLPDTEK are identical.

FIG. 4: MS analysis of a protein mixture following derivatization with Examples 18 and 19 in one single mixture. It can be seen from the ion chromatograms (FIG. 4a)) that the two variants elute at precisely the same time and appear with equal intensities. Apart from the expected shifts of 6 Da, the fragment spectra of the light (FIG. 4b)) and heavy (FIG. 4c)) variants of the labeled peptide APILSDSSCK are identical.

1. An organic compound of the formula (I),
A-L-PRG (I)
in which
A is an affinity ligand residue,
PRG is a protein-reactive group, and
L is a linker which covalently links A and PRG,
characterized in that the linker L contains an acid-cleavable group S of the formula 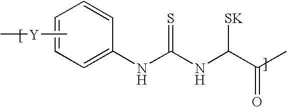
in which
Y is an optionally branched spacer group having a chain length of from 1 to 10, preferably of from 1 to 5, non-hydrogen atoms, and
SK is the side chain of an amino acid,
or the salt thereof.

2. A compound as claimed in claim 1, characterized in that the spacer group Y is bonded to the benzene ring in the para position in relation to the nitrogen atom.

3. A compound as claimed in claim 1 or 2, characterized in that the spacer group Y is a chain constructed from the optionally substituted building blocks NH, CH₂ and/or CO, preferably possesses at least NH group and is selected, in particular, from NH, NH—CH₂ and NH—CH₂—CH₂—NH—CO.

4. A compound as claimed in one of the preceding claims 1 to 3, characterized in that the protein-reactive group PRG exhibits a selective reactivity for one or more terminal functional groups of an amino acid group or phosphate group in the protein and/or for aldehyde or keto groups which are generated therefrom.

5. A compound as claimed in one of the preceding claims, characterized in that the protein-reactive group—PRG is selected from

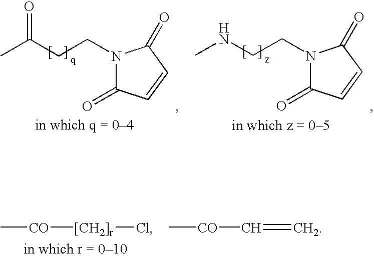

6. A compound as claimed in one of the preceding claims, characterized in that the affinity ligand residue A is the residue of biotin, of a biotin derivative, of a carbohydrate, of a hapten, of a sequestering agent or of a functional group which enables the compound to be covalently attached to a polymeric matrix, in particular of biotin or of a biotin derivative.

7. A compound as claimed in one of the preceding claims, characterized in that, for the isotope coding, it is isotope-labeled with at least one carbon atom of the isotope ¹³C, preferably from four to 15 ¹³C atoms.

8. A compound as claimed in the preceding claim, characterized in that, for the isotope coding, it is additionally isotope-labeled with at least one nitrogen atom of the isotope ¹⁵N, preferably from one to three ¹⁵N atoms, in particular one ¹⁵N atom.

9. A compound as claimed in one of the preceding claims, characterized in that it is a compound of the formula (II)
A-S—B¹—X¹—(CH₂)ₙ—[X²—(CH₂)ₘ]ₓ—X³—(CH₂)ₚ—X⁴—PRG (II)
in which
A is the affinity ligand residue,
PRG is the protein-reactive group,
S is the acid-cleavable group whose spacer group Y is preferably bonded to the benzene ring in the para position in relation to the nitrogen atom and is particularly preferably NH, NH—CH₂ or NH—CH₂—CH₂—NH—CO,
X¹, X² and X³ are, independently of each other, and with each X², being independently of other X², O, S, NH, NR, CO, CO—O, O—CO, CO—S, S—CO, S—S, SO, SO₂, CO—NR, NR—CO, CS—NR, NR—CS, Si—O or O—Si or an arylene or diarylene group, or a single bond,
X⁴ is O, S, NH, NR, CO—NR, NR—CO, CS—NR or NR—CS, or an arylene or diarylene group, preferably NH, NR, O or S,
m, n, p and x are, independently of each other, in each case a number from 0 to 100, where the sum n+xm+p is preferably less than 100 and particularly preferably between 10 and 30,
B¹ is an optional amine group NRR′ having the connectivity S—NRR′—X¹, and
R and R′ are, independently of each other, in each case hydrogen or alkyl, alkenyl, alkynyl, alkoxy or aryl, or are both, together with N, an N heterocycle NRR′, where R′ is additionally bonded to X¹ and cannot therefore be hydrogen.

10. A compound as claimed in one of the preceding claims, characterized in that it possesses a protein-reactive group PRG as claimed in claim 5 and a linker L as claimed in claim 9, where the sum of n+xm+p and q, z, r or s is preferably less than 100 and particularly preferably between 5 and 30.

11. A process for preparing a compound of the formula (II) as claimed in claim 9 or 10, in which
i) a compound of the formula (III)
H—B¹—X¹—(CH₂)ₙ—[X²—(CH₂)ₘ]ₓ—X³—(CH₂)ₚ—X⁴—V (II)
in which V is a hydrogen atom or a hydroxyl group and the other variables have the same meaning as in formula (II),
is initially reacted with an amino acid derivative which is protected at the N-terminal amino group to give a conjugate of the formula (IV),
SG-NH—CH(SK)—CO—B¹—X¹—(CH₂)ₙ—[X²—(CH₂)ₘ]ₓ—X³—(CH₂)ₚ—X⁴—V (IV)
in which SG is a protecting group and SK is the side chain of an amino acid,
ii) the conjugate of the formula (IV) is then reacted with the derivative of a protein-reactive group or the activated precursor of a protein-reactive group of the formula (V),
U—PRG (V)
in which U is a group which enables PRG to be linked to X⁴ by, for example, the group becoming a leaving group together with the residue V of the conjugate of the formula (IV),
iii) the amino protecting group SG is then detached, with a conjugate of the formula (VI)
H₂N—CH(SK)—CO—B¹—X¹—(CH₂)ₙ—[X²—(CH₂)ₘ]ₓ—X³—(CH₂)ₚ—X⁴—PRG (VI)
being obtained,
iv) the affinity ligand A-OH, or an activated form thereof, is reacted with a compound of the formula (VII), 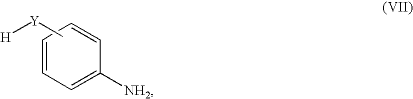
in which Y is the optionally branched spacer group,
which can optionally carry a protecting group, to give the compound of the formula (VIII) 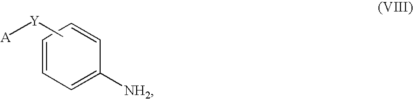
v) the compound of the formula (VIII) is then, after prior elimination of an optionally introduced protecting group, converted into a corresponding isothiocyanate,
vi) the isothiocyanate is then coupled to the conjugate of the formula (VI) to give the thiourea of the formula (IX), and 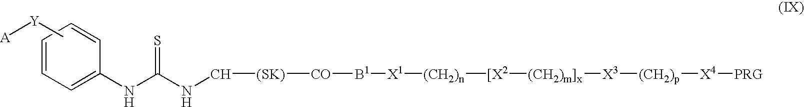
vii) in an optional last step, any protecting groups which may possibly still be present are eliminated,
with it being possible to carry out the consecutive steps iv) and v) at any arbitrary time prior to step vi).

12. A process for preparing a compound of the formula (II) as claimed in claim 10, in which
i) a compound of the formula (III)
H—B¹—X¹—(CH₂)ₙ—[X²—(CH₂)ₘ—X³—(CH₂)ₚ—X⁴—V (III)
in which V is a hydrogen atom or a hydroxyl group and the other variables have the same meaning as in formula (II),
is initially reacted with an amino acid derivative which is protected at the N-terminal amino group to give a conjugate of the formula (IV),
SG-NH—CH(SK)—CO—B¹—X¹—(CH₂)ₙ—[X²—(CH₂)ₘ]ₓ—X³—(CH₂)ₚ—X⁴—V (IV)
in which SG is a protecting group and SK is the side chain of an amino acid,
ii) the conjugate of the formula (IV) is converted, by introducing a further protecting group SG′, into the derivative of the formula (XIII),
SG-NH—CH(SK)—CO—B¹—X¹—(CH₂)ₙ—[X²—(CH₂)ₘ]ₓ—X³—(CH₂)ₚ—X⁴—SG′ (XIII)
and the N-terminal protecting group SG is then detached,
iii) the affinity ligand A-OH, or an activated form thereof, is reacted with a compound of the formula (VII) 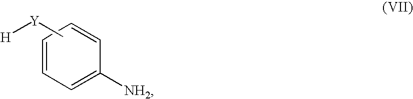
in which Y is the optionally branched spacer group,
which can optionally carry a protecting group, to give the compound of the formula (VII) 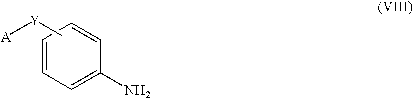
iv) the compound of the formula (VIII) is then converted, after prior elimination of an optionally introduced protecting group, into a corresponding isothiocyanate,
v) the isothiocyanate is reacted with the derivative of the formula (XIII) to give the conjugate of the formula (XIV)
A-S—B¹—X¹—(CH₂)ₙ—[X²—(CH₂)ₘ]ₓ—X³—(CH₂)ₚ—X⁴—SG′ (XIV).
vi) the conjugate of the formula (XIV) is coupled, after the protecting group SG′ has been detached, to the derivative of a protein-reactive group or the activated precursor of a protein-reactive group of the formula (V)
U—PRG (V)
in which U is a group which enables PRG to be linked to X⁴ by, for example, becoming a leaving group together with the residue V of the conjugate of the formula (IV),
to give the thiourea of the formula (IX), and 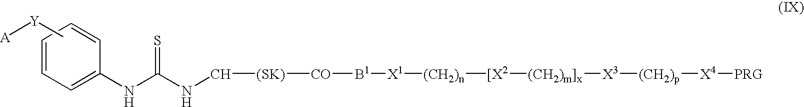
vii) in an optional last step, any protecting groups which may possibly still be present are eliminated,
with it being possible to carry out the consecutive steps iii) and iv) at any arbitrary time prior to step v).

13. The use of one or more differently isotope-labeled compounds as claimed in one of the preceding claims as (a) reagent(s) for the mass-spectrometric analysis of proteins.

14. The use as claimed in the preceding claim for identifying one or more proteins or protein functions in one or more protein-containing samples.

15. The use as claimed in claim 13 for determining the relative level of expression of one or more proteins in one or more protein-containing samples.
